SEQUENCE LISTING (446 sequences)

SEQ ID NO: 1 - DNA, 195 bp, artificial sequence
  Synthetic gene
ggtcgtttccacgaaaactggccgtctggtggcggtacctctacttccaaagcttccaccactacgacttctagcaaaaccaccactacatcctctaagactaccacgactacctccaaaacctctactacctctagctcctctacgggcggtggcactcacaagacctctactcagcgtctgctggctgcataa

SEQ ID NO: 2 - protein, 68 aa, artificial sequence
  Synthetic triblock peptide
GSIEGRFHENWPSAGGTSTSKASTTTTSSKTTTTSSKTTTTTSKTSTTSSSSTGGATHKTSTQRLLAA

SEQ ID NO: 3 - DNA, 103 bp, artificial sequence
  oligo used to prepare TBP101
ggatccatcgaaggtcgtttccacgaaaactggccgtctggtggcggtacctctacttccaaagcttccaccactacgacttctagcaaaaccaccactacat

SEQ ID NO: 4 - DNA, 104 bp, artificial sequence
  oligo used to prepare TBP101
cctctaagactaccacgactacctccaaaacctctactacctctagctcctctacgggcggtggcactcacaagacctctactcagcgtctgctggctgcataa

SEQ ID NO: 5 - DNA, 69 bp, artificial sequence
  oligo used to prepare TBP101
ttatgcagccagcagacgctgagtagaggtcttgtgagtgccaccgcccgtagaggagctagaggtagt

SEQ ID NO: 6 - DNA, 69 bp, artificial sequence
  oligo used to prepare TBP101
agaggttttggaggtagtcgtggtagtcttagaggatgtagtggtggttttgctagaagtcgtagtggt

SEQ ID NO: 7 - DNA, 69 bp, artificial sequence
  oligo used to prepare TBP101
ggaagctttggaagtagaggtaccgccaccagacggccagttttcgtggaaacgaccttcgatggatcc

SEQ ID NO: 8 - DNA, 22 bp, artificial sequence
  Primer
caccggatccatcgaaggtcgt

SEQ ID NO: 9 - DNA, 21 bp, artificial sequence
  Primer
tcattatgcagccagcagcgc

SEQ ID NO: 10 - DNA, 2580 bp, artificial sequence
  Plasmid
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 11 - DNA, 6354 bp, artificial sequence
  Plasmid
agatctcgatcccgcgaaattaatacgactcactatagggagaccacaacggtttccctctagaaataattttgtttaactttaagaaggagatatacatatgtcgtactaccatcaccatcaccatcacctcgaatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 12 - DNA, 294 bp, artificial sequence
  synthetic gene
atgtcgtactaccatcaccatcaccatcacctcgaatcaacaagtttgtacaaaaaagcaggctccgcggccgcccccttcaccggatccatcgaaggtcgtttccacgaaaactggccgtctgccggcggtacctctacttccaaagcttccaccactacgacttctagcaaaaccaccactacatcctctaagactaccacgactacctccaaaacctctactacctctagctcctctacgggcggcgccactcacaagacctctactcagcgtctgctggctgcataatga

SEQ ID NO: 13 - protein, 96 aa, artificial sequence
  synthetic peptide
MSYYHHHHHHLESTSLYKKAGSAAAPFTGSIEGRFHENWPSAGGTSTSKASTTTTSSKTTTTSSKTTTTTSKTSTTSSSSTGGATHKTSTQRLLAA

SEQ ID NO: 14 - DNA, 4945 bp, artificial sequence
  Plasmid
agatctcgatcccgcgaaattaatacgactcactatagggagaccacaacggtttccctctagaaataattttgtttaactttaagaaggagatatacatatgtcgtactaccatcaccatcaccatcacctcgaatcaacaagtttgtacaaaaaagcaggctccgcggccgcccccttcaccggatccatcgatccacgtttccacgaaaactggccgtctgccggcggtacctctacttccaaagcttccaccactacgacttctagcaaaaccaccactacatcctctaagactaccacgactacctccaaaacctctactacctctagctcctctacgggcggcgccactcacaagacctctactcagcgtctgctggctgcataatgaaagggtgggcgcgccgacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 15 - DNA, 45 bp, artificial sequence
  synthetic oligonucleotide
ccccttcaccggatccatcgatccacgtttccacgaaaactggcc

SEQ ID NO: 16 - DNA, 45 bp, artificial sequence
  synthetic oligonucleotide
ggccagttttcgtggaaacgtggatcgatggatccggtgaagggg

SEQ ID NO: 17 - DNA, 294 bp, artificial sequence
  synthetic gene
atgtcgtactaccatcaccatcaccatcacctcgaatcaacaagtttgtacaaaaaagcaggctccgcggccgcccccttcaccggatccatcgatccacgtttccacgaaaactggccgtctgccggcggtacctctacttccaaagcttccaccactacgacttctagcaaaaccaccactacatcctctaagactaccacgactacctccaaaacctctactacctctagctcctctacgggcggcgccactcacaagacctctactcagcgtctgctggctgcataatga

SEQ ID NO: 18 - protein, 96 aa, artificial sequence
  synthetic peptide
MSYYHHHHHHLESTSLYKKAGSAAAPFTGSIDPRFHENWPSAGGTSTSKASTTTTSSKTTTTSSKTTTTTSKTSTTSSSSTGGATHKTSTQRLLAA

SEQ ID NO: 19 - DNA, 672 bp, Zea mays
atgagggtgttgctcgttgccctcgctctcctggctctcgctgcgagcgccacctccacgcatacaagcggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccagctgcagggaacctgcggcgttggcagcaccccgatcctgggccagtgcgtcgagttcctgaggcatcagtgcagcccgacggcgacgccctactgctcgcctcagtgccagtcgttgcggcagcagtgttgccagcagctcaggcaggtggagccgcagcaccggtaccaggcgatcttcggcttggtcctccagtccatcctgcagcagcagccgcaaagcggccaggtcgcggggctgttggcggcgcagatagcgcagcaactgacggcgatgtgcggcctgcagcagccgactccatgcccctacgctgctgccggcggtgtcccccactga

SEQ ID NO: 20 - protein, 223 aa, Zea mays
MRVLLVALALLALAASATSTHTSGGCGCQPPPPVHLPPPVHLPPPVHLPPPVHLPPPVHLPPPVHLPPPVHVPPPVHLPPPPCHYPTQPPRPQPHPQPHPCPCQQPHPSPCQLQGTCGVGSTPILGQCVEFLRHQCSPTATPYCSPQCQSLRQQCCQQLRQVEPQHRYQAIFGLVLQSILQQQPQSGQVAGLLAAQIAQQLTAMCGLQQPTPCPYAAAGGVPH

SEQ ID NO: 21 - DNA, 47 bp, artificial sequence
  synthetic oligonucleotide
tatgcgtgtgctgctggtggcgctggcgctgctggcgctggcggcgg

SEQ ID NO: 22 - DNA, 49 bp, artificial sequence
  synthetic oligonucleotide
gatcccgccgccagcgccagcagcgccagcgccaccagcagcacacgca

SEQ ID NO: 23 - DNA, 50 bp, artificial sequence
  synthetic oligonucleotide
tatgagcgcgaccagcacccacaccagcggtggttgcggttgccagccgg

SEQ ID NO: 24 - DNA, 52 bp, artificial sequence
  synthetic oligonucleotide
gatcccggctggcaaccgcaaccaccgctggtgtgggtgctggtcgcgctca

SEQ ID NO: 25 - DNA, 50 bp, artificial sequence
  synthetic oligonucleotide
tatgccgccgccggtgcacctgccgccgccggtgcacctgccgccgccgg

SEQ ID NO: 26 - DNA, 52 bp, artificial sequence
  synthetic oligonucleotide
gatcccggcggcggcaggtgcaccggcggcggcaggtgcaccggcggcggca

SEQ ID NO: 27 - DNA, 50 bp, artificial sequence
  synthetic oligonucleotide
tatggtgcacctgccgccgccggtgcacctgccgccgccggtgcacctgg

SEQ ID NO: 28 - DNA, 52 bp, artificial sequence
  synthetic oligonucleotide
gatcccaggtgcaccggcggcggcaggtgcaccggcggcggcaggtgcacca

SEQ ID NO: 29 - DNA, 50 bp, artificial sequence
  synthetic oligonucleotide
tatgccgccgccggtgcacctgccgccgccggtgcacgtgccgccgccgg

SEQ ID NO: 30 - DNA, 52 bp, artificial sequence
  synthetic oligonucleotide
gatcccggcggcggcacgtgcaccggcggcggcaggtgcaccggcggcggca

SEQ ID NO: 31 - DNA, 50 bp, artificial sequence
  synthetic oligonucleotide
tatggtgcacctgccgccgccgccgtgccactacccgacccagccgccgg

SEQ ID NO: 32 - DNA, 52 bp, artificial sequence
  synthetic oligonucleotide
gatcccggcggctgggtcgggtagtggcacggcggcggcggcaggtgcacca

SEQ ID NO: 33 - DNA, 50 bp, artificial sequence
  synthetic oligonucleotide
tatgcgtccgcagccgcacccgcagccgcacccgtgcccgtgccagcagg

SEQ ID NO: 34 - DNA, 52 bp, artificial sequence
  synthetic oligonucleotide
gatccctgctggcacgggcacgggtgcggctgcgggtgcggctgcggacgca

SEQ ID NO: 35 - DNA, 50 bp, artificial sequence
  synthetic oligonucleotide
tatgccgcacccgagcccgtgccagctgcaggggacctgcggtgtgggtg

SEQ ID NO: 36 - DNA, 52 bp, artificial sequence
  synthetic oligonucleotide
gatccacccacaccgcaggtcccctgcagctggcacgggctcgggtgcggca

SEQ ID NO: 37 - DNA, 50 bp, artificial sequence
  synthetic oligonucleotide
tatgagcaccccgatcctgggtcagtgcgtggaattcctgcgtcaccagg

SEQ ID NO: 38 - DNA, 52 bp, artificial sequence
  synthetic oligonucleotide
gatccctggtgacgcaggaattccacgcactgacccaggatcggggtgctca

SEQ ID NO: 39 - DNA, 50 bp, artificial sequence
  synthetic oligonucleotide
tatgtgcagcccgaccgcgaccccgtactgcagcccgcagtgccagagcg

SEQ ID NO: 40 - DNA, 52 bp, artificial sequence
  synthetic oligonucleotide
gatccgctctggcactgcgggctgcagtacggggtcgcggtcgggctgcaca

SEQ ID NO: 41 - DNA, 50 bp, artificial sequence
  synthetic oligonucleotide
tatgctgcgtcagcagtgctgccagcagctgcgtcaggtggaaccgcagg

SEQ ID NO: 42 - DNA, 52 bp, artificial sequence
  synthetic oligonucleotide
gatccctgcggttccacctgacgcagctgctggcagcactgctgacgcagca

SEQ ID NO: 43 - DNA, 50 bp, artificial sequence
  synthetic oligonucleotide
tatgcaccgttaccaggcgatcttcggtctggtgctgcagagcatcctgg

SEQ ID NO: 44 - DNA, 52 bp, artificial sequence
  synthetic oligonucleotide
gatcccaggatgctctgcagcaccagaccgaagatcgcctggtaacggtgca

SEQ ID NO: 45 - DNA, 50 bp, artificial sequence
  synthetic oligonucleotide
tatgcagcagcagccgcagagcggtcaggtggcgggtctgctggcggcgg

SEQ ID NO: 46 - DNA, 52 bp, artificial sequence
  synthetic oligonucleotide
gatcccgccgccagcagacccgccacctgaccgctctgcggctgctgctgca

SEQ ID NO: 47 - DNA, 50 bp, artificial sequence
  synthetic oligonucleotide
tatgcagatcgcgcagcagctgaccgcgatgtgcggtctgcagcagccgg

SEQ ID NO: 48 - DNA, 52 bp, artificial sequence
  synthetic oligonucleotide
gatcccggctgctgcagaccgcacatcgcggtcagctgctgcgcgatctgca

SEQ ID NO: 49 - DNA, 44 bp, artificial sequence
  synthetic oligonucleotide
tatgaccccgtgcccgtacgcggcggcgggtggtgtgccgcacg

SEQ ID NO: 50 - DNA, 46 bp, artificial sequence
  synthetic oligonucleotide
gatccgtgcggcacaccacccgccgccgcgtacgggcacggggtca

SEQ ID NO: 51 - DNA, 50 bp, artificial sequence
  synthetic oligonucleotide
tatggtggcgctggcgctgctggcgctggcggcgagcgcgaccagcaccg

SEQ ID NO: 52 - DNA, 52 bp, artificial sequence
  synthetic oligonucleotide
gatccggtgctggtcgcgctcgccgccagcgccagcagcgccagcgccacca

SEQ ID NO: 53 - DNA, 50 bp, artificial sequence
  synthetic oligonucleotide
tatgcacaccagcggtggttgcggttgccagccgccgccgccggtgcacg

SEQ ID NO: 54 - DNA, 52 bp, artificial sequence
  synthetic oligonucleotide
gatccgtgcaccggcggcggcggctggcaaccgcaaccaccgctggtgtgca

SEQ ID NO: 55 - DNA, 50 bp, artificial sequence
  synthetic oligonucleotide
tatgctgccgccgccggtgcacctgccgccgccggtgcacctgccgccgg

SEQ ID NO: 56 - DNA, 52 bp, artificial sequence
  synthetic oligonucleotide
gatcccggcggcaggtgcaccggcggcggcaggtgcaccggcggcggcagca

SEQ ID NO: 57 - DNA, 50 bp, artificial sequence
  synthetic oligonucleotide
tatgccggtgcacctgccgccgccggtgcacctgccgccgccggtgcacg

SEQ ID NO: 58 - DNA, 52 bp, artificial sequence
  synthetic oligonucleotide
gatccgtgcaccggcggcggcaggtgcaccggcggcggcaggtgcaccggca

SEQ ID NO: 59 - DNA, 50 bp, artificial sequence
  synthetic oligonucleotide
tatgctgccgccgccggtgcacgtgccgccgccggtgcacctgccgccgg

SEQ ID NO: 60 - DNA, 52 bp, artificial sequence
  synthetic oligonucleotide
gatcccggcggcaggtgcaccggcggcggcacgtgcaccggcggcggcagca

SEQ ID NO: 61 - DNA, 50 bp, artificial sequence
  synthetic oligonucleotide
tatgccgccgtgccactacccgacccagccgccgcgtccgcagccgcacg

SEQ ID NO: 62 - DNA, 52 bp, artificial sequence
  synthetic oligonucleotide
gatccgtgcggctgcggacgcggcggctgggtcgggtagtggcacggcggca

SEQ ID NO: 63 - DNA, 50 bp, artificial sequence
  synthetic oligonucleotide
tatgccgcagccgcacccgtgcccgtgccagcagccgcacccgagcccgg

SEQ ID NO: 64 - DNA, 52 bp, artificial sequence
  synthetic oligonucleotide
gatcccgggctcgggtgcggctgctggcacgggcacgggtgcggctgcggca

SEQ ID NO: 65 - DNA, 50 bp, artificial sequence
  synthetic oligonucleotide
tatgtgccagctgcaggggacctgcggtgtgggtagcaccccgatcctgg

SEQ ID NO: 66 - DNA, 52 bp, artificial sequence
  synthetic oligonucleotide
gatcccaggatcggggtgctacccacaccgcaggtcccctgcagctggcaca

SEQ ID NO: 67 - DNA, 50 bp, artificial sequence
  synthetic oligonucleotide
tatgggtcagtgcgtggaattcctgcgtcaccagtgcagcccgaccgcgg

SEQ ID NO: 68 - DNA, 52 bp, artificial sequence
  synthetic oligonucleotide
gatcccgcggtcgggctgcactggtgacgcaggaattccacgcactgaccca

SEQ ID NO: 69 - DNA, 50 bp, artificial sequence
  synthetic oligonucleotide
tatgaccccgtactgcagcccgcagtgccagagcctgcgtcagcagtgcg

SEQ ID NO: 70 - DNA, 52 bp, artificial sequence
  synthetic oligonucleotide
gatccgcactgctgacgcaggctctggcactgcgggctgcagtacggggtca

SEQ ID NO: 71 - DNA, 50 bp, artificial sequence
  synthetic oligonucleotide
tatgtgccagcagctgcgtcaggtggaaccgcagcaccgttaccaggcgg

SEQ ID NO: 72 - DNA, 52 bp, artificial sequence
  synthetic oligonucleotide
gatcccgcctggtaacggtgctgcggttccacctgacgcagctgctggcaca

SEQ ID NO: 73 - DNA, 50 bp, artificial sequence
  synthetic oligonucleotide
tatgatcttcggtctggtgctgcagagcatcctgcagcagcagccgcagg

SEQ ID NO: 74 - DNA, 52 bp, artificial sequence
  synthetic oligonucleotide
gatccctgcggctgctgctgcaggatgctctgcagcaccagaccgaagatca

SEQ ID NO: 75 - DNA, 50 bp, artificial sequence
  synthetic oligonucleotide
tatgagcggtcaggtggcgggtctgctggcggcgcagatcgcgcagcagg

SEQ ID NO: 76 - DNA, 52 bp, artificial sequence
  synthetic oligonucleotide
gatccctgctgcgcgatctgcgccgccagcagacccgccacctgaccgctca

SEQ ID NO: 77 - DNA, 50 bp, artificial sequence
  synthetic oligonucleotide
tatgctgaccgcgatgtgcggtctgcagcagccgaccccgtgcccgtacg

SEQ ID NO: 78 - DNA, 52 bp, artificial sequence
  synthetic oligonucleotide
gatccgtacgggcacggggtcggctgctgcagaccgcacatcgcggtcagca

SEQ ID NO: 79 - DNA, 50 bp, artificial sequence
  synthetic oligonucleotide
tatgctggcgctggcggcgagcgcgaccagcacccacaccagcggtggtg

SEQ ID NO: 80 - DNA, 52 bp, artificial sequence
  synthetic oligonucleotide
gatccaccaccgctggtgtgggtgctggtcgcgctcgccgccagcgccagca

SEQ ID NO: 81 - DNA, 50 bp, artificial sequence
  synthetic oligonucleotide
tatgtgcggttgccagccgccgccgccggtgcacctgccgccgccggtgg

SEQ ID NO: 82 - DNA, 52 bp, artificial sequence
  synthetic oligonucleotide
gatcccaccggcggcggcaggtgcaccggcggcggcggctggcaaccgcaca

SEQ ID NO: 83 - DNA, 50 bp, artificial sequence
  synthetic oligonucleotide
tatgcacctgccgccgccggtgcacctgccgccgccggtgcacctgccgg

SEQ ID NO: 84 - DNA, 52 bp, artificial sequence
  synthetic oligonucleotide
gatcccggcaggtgcaccggcggcggcaggtgcaccggcggcggcaggtgca

SEQ ID NO: 85 - DNA, 50 bp, artificial sequence
  synthetic oligonucleotide
tatgccgccggtgcacctgccgccgccggtgcacctgccgccgccggtgg

SEQ ID NO: 86 - DNA, 52 bp, artificial sequence
  synthetic oligonucleotide
gatcccaccggcggcggcaggtgcaccggcggcggcaggtgcaccggcggca

SEQ ID NO: 87 - DNA, 50 bp, artificial sequence
  synthetic oligonucleotide
tatgcacgtgccgccgccggtgcacctgccgccgccgccgtgccactacg

SEQ ID NO: 88 - DNA, 52 bp, artificial sequence
  synthetic oligonucleotide
gatccgtagtggcacggcggcggcggcaggtgcaccggcggcggcacgtgca

SEQ ID NO: 89 - DNA, 50 bp, artificial sequence
  synthetic oligonucleotide
tatgccgacccagccgccgcgtccgcagccgcacccgcagccgcacccgg

SEQ ID NO: 90 - DNA, 52 bp, artificial sequence
  synthetic oligonucleotide
gatcccgggtgcggctgcgggtgcggctgcggacgcggcggctgggtcggca

SEQ ID NO: 91 - DNA, 50 bp, artificial sequence
  synthetic oligonucleotide
tatgtgcccgtgccagcagccgcacccgagcccgtgccagctgcaggggg

SEQ ID NO: 92 - DNA, 52 bp, artificial sequence
  synthetic oligonucleotide
gatcccccctgcagctggcacgggctcgggtgcggctgctggcacgggcaca

SEQ ID NO: 93 - DNA, 50 bp, artificial sequence
  synthetic oligonucleotide
tatgacctgcggtgtgggtagcaccccgatcctgggtcagtgcgtggaag

SEQ ID NO: 94 - DNA, 52 bp, artificial sequence
  synthetic oligonucleotide
gatccttccacgcactgacccaggatcggggtgctacccacaccgcaggtca

SEQ ID NO: 95 - DNA, 50 bp, artificial sequence
  synthetic oligonucleotide
tatgttcctgcgtcaccagtgcagcccgaccgcgaccccgtactgcagcg

SEQ ID NO: 96 - DNA, 52 bp, artificial sequence
  synthetic oligonucleotide
gatccgctgcagtacggggtcgcggtcgggctgcactggtgacgcaggaaca

SEQ ID NO: 97 - DNA, 50 bp, artificial sequence
  synthetic oligonucleotide
tatgccgcagtgccagagcctgcgtcagcagtgctgccagcagctgcgtg

SEQ ID NO: 98 - DNA, 52 bp, artificial sequence
  synthetic oligonucleotide
gatccacgcagctgctggcagcactgctgacgcaggctctggcactgcggca

SEQ ID NO: 99 - DNA, 50 bp, artificial sequence
  synthetic oligonucleotide
tatgcaggtggaaccgcagcaccgttaccaggcgatcttcggtctggtgg

SEQ ID NO: 100 - DNA, 52 bp, artificial sequence
  synthetic oligonucleotide
gatcccaccagaccgaagatcgcctggtaacggtgctgcggttccacctgca

SEQ ID NO: 101 - DNA, 50 bp, artificial sequence
  synthetic oligonucleotide
tatgctgcagagcatcctgcagcagcagccgcagagcggtcaggtggcgg

SEQ ID NO: 102 - DNA, 52 bp, artificial sequence
  synthetic oligonucleotide
gatcccgccacctgaccgctctgcggctgctgctgcaggatgctctgcagca

SEQ ID NO: 103 - DNA, 50 bp, artificial sequence
  synthetic oligonucleotide
tatgggtctgctggcggcgcagatcgcgcagcagctgaccgcgatgtgcg

SEQ ID NO: 104 - DNA, 52 bp, artificial sequence
  synthetic oligonucleotide
gatccgcacatcgcggtcagctgctgcgcgatctgcgccgccagcagaccca

SEQ ID NO: 105 - DNA, 50 bp, artificial sequence
  synthetic oligonucleotide
tatgggtctgcagcagccgaccccgtgcccgtacgcggcggcgggtggtg

SEQ ID NO: 106 - DNA, 52 bp, artificial sequence
  synthetic oligonucleotide
gatccaccacccgccgccgcgtacgggcacggggtcggctgctgcagaccca

SEQ ID NO: 107 - DNA, 80 bp, artificial sequence
  synthetic oligonucleotide
tatgccgacccagccgccgcgtccgcagccgcacccgcagccgcacccgtgcccgtgccagcagccgcacccgagcccgg

SEQ ID NO: 108 - DNA, 82 bp, artificial sequence
  synthetic oligonucleotide
gatcccgggctcgggtgcggctgctggcacgggcacgggtgcggctgcgggtgcggctgcggacgcggcggctgggtcggca

SEQ ID NO: 109 - DNA, 65 bp, artificial sequence
  synthetic oligonucleotide
tatgcgtccgcagccgcacccgcagccgcacccgtgcccgtgccagcagccgcacccgagcccgg

SEQ ID NO: 110 - DNA, 67 bp, artificial sequence
  synthetic oligonucleotide
gatcccgggctcgggtgcggctgctggcacgggcacgggtgcggctgcgggtgcggctgcggacgca

SEQ ID NO: 111 - protein, 15 aa, Zea mays
MRVLLVALALLALAA

SEQ ID NO: 112 - protein, 15 aa, Zea mays
SATSTHTSGGCGCQP

SEQ ID NO: 113 - protein, 15 aa, Zea mays
PPPVHLPPPVHLPPP

SEQ ID NO: 114 - protein, 15 aa, Zea mays
VHLPPPVHLPPPVHL

SEQ ID NO: 115 - protein, 15 aa, Zea mays
PPPVHLPPPVHVPPP

SEQ ID NO: 116 - protein, 15 aa, Zea mays
VHLPPPPCHYPTQPP

SEQ ID NO: 117 - protein, 15 aa, Zea mays
RPQPHPQPHPCPCQQ

SEQ ID NO: 118 - protein, 15 aa, Zea mays
PHPSPCQLQGTCGVG

SEQ ID NO: 119 - protein, 15 aa, Zea mays
STPILGQCVEFLRHQ

SEQ ID NO: 120 - protein, 15 aa, Zea mays
CSPTATPYCSPQCQS

SEQ ID NO: 121 - protein, 15 aa, Zea mays
LRQQCCQQLRQVEPQ

SEQ ID NO: 122 - protein, 15 aa, Zea mays
HRYQAIFGLVLQSIL

SEQ ID NO: 123 - protein, 15 aa, Zea mays
QQQPQSGQVAGLLAA

SEQ ID NO: 124 - protein, 15 aa, Zea mays
QIAQQLTAMCGLQQP

SEQ ID NO: 125 - protein, 13 aa, Zea mays
TPCPYAAAGGVPH

SEQ ID NO: 126 - protein, 15 aa, Zea mays
VALALLALAASATST

SEQ ID NO: 127 - protein, 15 aa, Zea mays
HTSGGCGCQPPPPVH

SEQ ID NO: 128 - protein, 15 aa, Zea mays
LPPPVHLPPPVHLPP

SEQ ID NO: 129 - protein, 15 aa, Zea mays
PVHLPPPVHLPPPVH

SEQ ID NO: 130 - protein, 15 aa, Zea mays
LPPPVHVPPPVHLPP

SEQ ID NO: 131 - protein, 15 aa, Zea mays
PPCHYPTQPPRPQPH

SEQ ID NO: 132 - protein, 15 aa, Zea mays
PQPHPCPCQQPHPSP

SEQ ID NO: 133 - protein, 15 aa, Zea mays
CQLQGTCGVGSTPIL

SEQ ID NO: 134 - protein, 15 aa, Zea mays
GQCVEFLRHQCSPTA

SEQ ID NO: 135 - protein, 15 aa, Zea mays
TPYCSPQCQSLRQQC

SEQ ID NO: 136 - protein, 15 aa, Zea mays
CQQLRQVEPQHRYQA

SEQ ID NO: 137 - protein, 15 aa, Zea mays
IFGLVLQSILQQQPQ

SEQ ID NO: 138 - protein, 15 aa, Zea mays
SGQVAGLLAAQIAQQ

SEQ ID NO: 139 - protein, 15 aa, Zea mays
LTAMCGLQQPTPCPY

SEQ ID NO: 140 - protein, 15 aa, Zea mays
LALAASATSTHTSGG

SEQ ID NO: 141 - protein, 15 aa, Zea mays
CGCQPPPPVHLPPPV

SEQ ID NO: 142 - protein, 15 aa, Zea mays
HLPPPVHLPPPVHLP

SEQ ID NO: 143 - protein, 15 aa, Zea mays
PPVHLPPPVHLPPPV

SEQ ID NO: 144 - protein, 15 aa, Zea mays
HVPPPVHLPPPPCHY

SEQ ID NO: 145 - protein, 15 aa, Zea mays
PTQPPRPQPHPQPHP

SEQ ID NO: 146 - protein, 15 aa, Zea mays
CPCQQPHPSPCQLQG

SEQ ID NO: 147 - protein, 15 aa, Zea mays
TCGVGSTPILGQCVE

SEQ ID NO: 148 - protein, 15 aa, Zea mays
FLRHQCSPTATPYCS

SEQ ID NO: 149 - protein, 15 aa, Zea mays
PQCQSLRQQCCQQLR

SEQ ID NO: 150 - protein, 15 aa, Zea mays
QVEPQHRYQAIFGLV

SEQ ID NO: 151 - protein, 15 aa, Zea mays
LQSILQQQPQSGQVA

SEQ ID NO: 152 - protein, 15 aa, Zea mays
GLLAAQIAQQLTAMC

SEQ ID NO: 153 - protein, 15 aa, Zea mays
GLQQPTPCPYAAAGG

SEQ ID NO: 154 - protein, 25 aa, Zea mays
PTQPPRPQPHPQPHPCPCQQPHPSP

SEQ ID NO: 155 - protein, 20 aa, Zea mays
RPQPHPQPHPCPCQQPHPSP

SEQ ID NO: 156 - protein, 11 aa, artificial sequence
  T7 Translation enhancer element
MASMTGGQQMG

SEQ ID NO: 157 - protein, 41 aa, artificial sequence
  IBT-180 tag
MASMTGGQQMGVHLPPPPCHYPTQPPRPQPHPQPHPCPCQQ

SEQ ID NO: 158 - protein, 111 aa, artificial sequence
  IBT-181 tag
MASMTGGQQMGVHLPPPPCHYPTQPPRPQPHPQPHPCPCQQPHPSPCQLQGTCGVGSTPILGQCVEFLRHQCSPTATPYCSPQCQSLRQQCCQQLRQVEPQHRYQAIFGLV

SEQ ID NO: 159 - DNA, 402 bp, Daucus carota
atggcagcaaaaacacaagcaatcttaattctcctcctcatctccgccgtcctcatcgcctccccggccgcaggcctaggcggctccggcgccgtcggcggccgcaccgaaatccccgacgtcgaatccaacgaggagatccaacaattaggcgaatattccgtcgaacagtacaatcaacagcatcacaacggcgacggcggcgacagcaccgacagcgccggcgatctcaagttcgtgaaggtcgtcgcggcggagaagcaggtagtggccggaattaagtattacttgaagatcgtcgcggcgaaaggcggacacaagaagaagttcgatgcggagatcgttgtgcaggcgtggaagaagacgaagcagttgatgagcttcgctccgtcgcacaattga

SEQ ID NO: 160 - protein, 133 aa, Daucus carota
MAAKTQAILILLLISAVLIASPAAGLGGSGAVGGRTEIPDVESNEEIQQLGEYSVEQYNQQHHNGDGGDSTDSAGDLKFVKVVAAEKQVVAGIKYYLKIVAAKGGHKKKFDAEIVVQAWKKTKQLMSFAPSHN

SEQ ID NO: 161 - DNA, 38 bp, artificial sequence
  synthetic oligonucleotide
tatggcggcgaaaacccaggcgatcctgatcctgctgg

SEQ ID NO: 162 - DNA, 40 bp, artificial sequence
  synthetic oligonucleotide
gatcccagcaggatcaggatcgcctgggttttcgccgcca

SEQ ID NO: 163 - DNA, 41 bp, artificial sequence
  synthetic oligonucleotide
tatgctgatcagcgcggtgctgatcgccagcccggcggccg

SEQ ID NO: 164 - DNA, 43 bp, artificial sequence
  synthetic oligonucleotide
gatccggccgccgggctggcgatcagcaccgcgctgatcagca

SEQ ID NO: 165 - DNA, 41 bp, artificial sequence
  synthetic oligonucleotide
tatgggtctgggtggcagcggtgcggtgggcggtcgtaccg

SEQ ID NO: 166 - DNA, 43 bp, artificial sequence
  synthetic oligonucleotide
gatccggtacgaccgcccaccgcaccgctgccacccagaccca

SEQ ID NO: 167 - DNA, 41 bp, artificial sequence
  synthetic oligonucleotide
tatggaaatcccggatgtggaaagcaacgaagaaatccagg

SEQ ID NO: 168 - DNA, 43 bp, artificial sequence
  synthetic oligonucleotide
gatccctggatttcttcgttgctttccacatccgggatttcca

SEQ ID NO: 169 - DNA, 41 bp, artificial sequence
  synthetic oligonucleotide
tatgcagctgggtgaatacagcgtggaacagtacaaccagg

SEQ ID NO: 170 - DNA, 43 bp, artificial sequence
  synthetic oligonucleotide
gatccctggttgtactgttccacgctgtattcacccagctgca

SEQ ID NO: 171 - DNA, 41 bp, artificial sequence
  synthetic oligonucleotide
tatgcagcaccacaacggtgatggtggtgatagcaccgatg

SEQ ID NO: 172 - DNA, 43 bp, artificial sequence
  synthetic oligonucleotide
gatccatcggtgctatcaccaccatcaccgttgtggtgctgca

SEQ ID NO: 173 - DNA, 41 bp, artificial sequence
  synthetic oligonucleotide
tatgagcgcgggtgatctgaaattcgtgaaagtggtggcgg

SEQ ID NO: 174 - DNA, 43 bp, artificial sequence
  synthetic oligonucleotide
gatcccgccaccactttcacgaatttcagatcacccgcgctca

SEQ ID NO: 175 - DNA, 41 bp, artificial sequence
  synthetic oligonucleotide
tatggcggaaaaacaggtggtggcgggtatcaaatactacg

SEQ ID NO: 176 - DNA, 43 bp, artificial sequence
  synthetic oligonucleotide
gatccgtagtatttgatacccgccaccacctgtttttccgcca

SEQ ID NO: 177 - DNA, 41 bp, artificial sequence
  synthetic oligonucleotide
tatgctgaaaatcgtggcggcgaaaggtggtcacaaaaaag

SEQ ID NO: 178 - DNA, 43 bp, artificial sequence
  synthetic oligonucleotide
gatccttttttgtgaccacctttcgccgccacgattttcagca

SEQ ID NO: 179 - DNA, 41 bp, artificial sequence
  synthetic oligonucleotide
tatgaaattcgatgcggaaatcgtggtgcaggcgtggaaag

SEQ ID NO: 180 - DNA, 43 bp, artificial sequence
  synthetic oligonucleotide
gatcctttccacgcctgcaccacgatttccgcatcgaatttca

SEQ ID NO: 181 - DNA, 44 bp, artificial sequence
  synthetic oligonucleotide
tatgaaaaccaaacagctgatgagcttcgcgccgagccacaacg

SEQ ID NO: 182 - DNA, 46 bp, artificial sequence
  synthetic oligonucleotide
gatccgttgtggctcggcgcgaagctcatcagctgtttggttttca

SEQ ID NO: 183 - DNA, 41 bp, artificial sequence
  synthetic oligonucleotide
tatgacccaggcgatcctgatcctgctgctgatcagcgcgg

SEQ ID NO: 184 - DNA, 43 bp, artificial sequence
  synthetic oligonucleotide
gatcccgcgctgatcagcagcaggatcaggatcgcctgggtca

SEQ ID NO: 185 - DNA, 41 bp, artificial sequence
  synthetic oligonucleotide
tatggtgctgatcgcgagcccggcggcgggtctgggtggtg

SEQ ID NO: 186 - DNA, 40 bp, artificial sequence
  synthetic oligonucleotide
gatccaccacccagacccgccgccgggctcgcgatcagca

SEQ ID NO: 187 - DNA, 41 bp, artificial sequence
  synthetic oligonucleotide
tatgagcggtgcggtgggtggtcgtaccgaaatcccggatg

SEQ ID NO: 188 - DNA, 43 bp, artificial sequence
  synthetic oligonucleotide
gatccatccgggatttcggtacgaccacccaccgcaccgctca

SEQ ID NO: 189 - DNA, 41 bp, artificial sequence
  synthetic oligonucleotide
tatggtggaaagcaacgaagaaatccagcagctgggtgaag

SEQ ID NO: 190 - DNA, 43 bp, artificial sequence
  synthetic oligonucleotide
gatccttcacccagctgctggatttcttcgttgctttccacca

SEQ ID NO: 191 - DNA, 41 bp, artificial sequence
  synthetic oligonucleotide
tatgtacagcgtggaacagtacaaccagcagcaccacaacg

SEQ ID NO: 192 - DNA, 43 bp, artificial sequence
  synthetic oligonucleotide
gatccgttgtggtgctgctggttgtactgttccacgctgtaca

SEQ ID NO: 193 - DNA, 41 bp, artificial sequence
  synthetic oligonucleotide
tatgggtgatggtggtgatagcaccgatagcgcgggtgatg

SEQ ID NO: 194 - DNA, 43 bp, artificial sequence
  synthetic oligonucleotide
gatccatcacccgcgctatcggtgctatcaccaccatcaccca

SEQ ID NO: 195 - DNA, 41 bp, artificial sequence
  synthetic oligonucleotide
tatgctgaaattcgtgaaagtggtggcggcggaaaaacagg

SEQ ID NO: 196 - DNA, 43 bp, artificial sequence
  synthetic oligonucleotide
gatccctgtttttccgccgccaccactttcacgaatttcagca

SEQ ID NO: 197 - DNA, 41 bp, artificial sequence
  synthetic oligonucleotide
tatggtggtggcgggtatcaaatactacctgaaaatcgtgg

SEQ ID NO: 198 - DNA, 43 bp, artificial sequence
  synthetic oligonucleotide
gatcccacgattttcaggtagtatttgatacccgccaccacca

SEQ ID NO: 199 - DNA, 41 bp, artificial sequence
  synthetic oligonucleotide
tatggcggcgaaaggtggtcacaaaaaaaaattcgatgcgg

SEQ ID NO: 200 - DNA, 43 bp, artificial sequence
  synthetic oligonucleotide
gatcccgcatcgaatttttttttgtgaccacctttcgccgcca

SEQ ID NO: 201 - DNA, 41 bp, artificial sequence
  synthetic oligonucleotide
tatggaaatcgtggtgcaggcgtggaaaaaaaccaaacagg

SEQ ID NO: 202 - DNA, 43 bp, artificial sequence
  synthetic oligonucleotide
gatccctgtttggtttttttccacgcctgcaccacgatttcca

SEQ ID NO: 203 - DNA, 41 bp, artificial sequence
  synthetic oligonucleotide
tatgctgatcctgctgctgatcagcgcggtgctgatcgcgg

SEQ ID NO: 204 - DNA, 43 bp, artificial sequence
  synthetic oligonucleotide
gatcccgcgatcagcaccgcgctgatcagcagcaggatcagca

SEQ ID NO: 205 - DNA, 41 bp, artificial sequence
  synthetic oligonucleotide
tatgagcccggcggcgggtctgggtggtagcggtgcggtgg

SEQ ID NO: 206 - DNA, 43 bp, artificial sequence
  synthetic oligonucleotide
gatcccaccgcaccgctaccacccagacccgccgccgggctca

SEQ ID NO: 207 - DNA, 41 bp, artificial sequence
  synthetic oligonucleotide
tatgggtggtcgtaccgaaatcccggatgtggaaagcaacg

SEQ ID NO: 208 - DNA, 43 bp, artificial sequence
  synthetic oligonucleotide
gatccgttgctttccacatccgggatttcggtacgaccaccca

SEQ ID NO: 209 - DNA, 41 bp, artificial sequence
  synthetic oligonucleotide
tatggaagaaatccagcagctgggtgaatacagcgtggaag

SEQ ID NO: 210 - DNA, 43 bp, artificial sequence
  synthetic oligonucleotide
gatccttccacgctgtattcacccagctgctggatttcttcca

SEQ ID NO: 211 - DNA, 41 bp, artificial sequence
  synthetic oligonucleotide
tatgcagtacaaccagcagcaccacaacggtgatggtggtg

SEQ ID NO: 212 - DNA, 43 bp, artificial sequence
  synthetic oligonucleotide
gatccaccaccatcaccgttgtggtgctgctggttgtactgca

SEQ ID NO: 213 - DNA, 41 bp, artificial sequence
  synthetic oligonucleotide
tatggatagcaccgatagcgcgggtgatctgaaattcgtgg

SEQ ID NO: 214 - DNA, 43 bp, artificial sequence
  synthetic oligonucleotide
gatcccacgaatttcagatcacccgcgctatcggtgctatcca

SEQ ID NO: 215 - DNA, 41 bp, artificial sequence
  synthetic oligonucleotide
tatgaaagtggtggcggcggaaaaacaggtggtggcgggtg

SEQ ID NO: 216 - DNA, 43 bp, artificial sequence
  synthetic oligonucleotide
gatccacccgccaccacctgtttttccgccgccaccactttca

SEQ ID NO: 217 - DNA, 41 bp, artificial sequence
  synthetic oligonucleotide
tatgatcaaatactacctgaaaatcgtggcggcgaaaggtg

SEQ ID NO: 218 - DNA, 43 bp, artificial sequence
  synthetic oligonucleotide
gatccacctttcgccgccacgattttcaggtagtatttgatca

SEQ ID NO: 219 - DNA, 41 bp, artificial sequence
  synthetic oligonucleotide
tatgggtcacaaaaaaaaattcgatgcggaaatcgtggtgg

SEQ ID NO: 220 - DNA, 43 bp, artificial sequence
  synthetic oligonucleotide
gatcccaccacgatttccgcatcgaatttttttttgtgaccca

SEQ ID NO: 221 - DNA, 41 bp, artificial sequence
  synthetic oligonucleotide
tatgcaggcgtggaaaaaaaccaaacagctgatgagcttcg

SEQ ID NO: 222 - DNA, 43 bp, artificial sequence
  synthetic oligonucleotide
gatccgaagctcatcagctgtttggtttttttccacgcctgca

SEQ ID NO: 223 - protein, 12 aa, Daucus carota
MAAKTQAILILL

SEQ ID NO: 224 - protein, 12 aa, Daucus carota
LISAVLIASPAA

SEQ ID NO: 225 - protein, 12 aa, Daucus carota
GLGGSGAVGGRT

SEQ ID NO: 226 - protein, 12 aa, Daucus carota
EIPDVESNEEIQ

SEQ ID NO: 227 - protein, 12 aa, Daucus carota
QLGEYSVEQYNQ

SEQ ID NO: 228 - protein, 12 aa, Daucus carota
QHHNGDGGDSTD

SEQ ID NO: 229 - protein, 12 aa, Daucus carota
SAGDLKFVKVVA

SEQ ID NO: 230 - protein, 12 aa, Daucus carota
AEKQVVAGIKYY

SEQ ID NO: 231 - protein, 12 aa, Daucus carota
LKIVAAKGGHKK

SEQ ID NO: 232 - protein, 12 aa, Daucus carota
KFDAEIVVQAWK

SEQ ID NO: 233 - protein, 13 aa, Daucus carota
KTKQLMSFAPSHN

SEQ ID NO: 234 - protein, 12 aa, Daucus carota
TQAILILLLISA

SEQ ID NO: 235 - protein, 12 aa, Daucus carota
VLIASPAAGLGG

SEQ ID NO: 236 - protein, 12 aa, Daucus carota
SGAVGGRTEIPD

SEQ ID NO: 237 - protein, 12 aa, Daucus carota
VESNEEIQQLGE

SEQ ID NO: 238 - protein, 12 aa, Daucus carota
YSVEQYNQQHHN

SEQ ID NO: 239 - protein, 12 aa, Daucus carota
GDGGDSTDSAGD

SEQ ID NO: 240 - protein, 12 aa, Daucus carota
LKFVKVVAAEKQ

SEQ ID NO: 241 - protein, 12 aa, Daucus carota
VVAGIKYYLKIV

SEQ ID NO: 242 - protein, 12 aa, Daucus carota
AAKGGHKKKFDA

SEQ ID NO: 243 - protein, 12 aa, Daucus carota
EIVVQAWKKTKQ

SEQ ID NO: 244 - protein, 12 aa, Daucus carota
LILLLISAVLIA

SEQ ID NO: 245 - protein, 12 aa, Daucus carota
SPAAGLGGSGAV

SEQ ID NO: 246 - protein, 12 aa, Daucus carota
GGRTEIPDVESN

SEQ ID NO: 247 - protein, 12 aa, Daucus carota
EEIQQLGEYSVE

SEQ ID NO: 248 - protein, 12 aa, Daucus carota
QYNQQHHNGDGG

SEQ ID NO: 249 - protein, 12 aa, Daucus carota
DSTDSAGDLKFV

SEQ ID NO: 250 - protein, 12 aa, Daucus carota
KVVAAEKQVVAG

SEQ ID NO: 251 - protein, 12 aa, Daucus carota
IKYYLKIVAAKG

SEQ ID NO: 252 - protein, 12 aa, Daucus carota
GHKKKFDAEIVV

SEQ ID NO: 253 - protein, 12 aa, Daucus carota
QAWKKTKQLMSF

SEQ ID NO: 254 - protein, 7 aa, artificial sequence
  skin binding peptide
FTQSLPR

SEQ ID NO: 255 - protein, 12 aa, artificial sequence
  skin binding peptide
TPFHSPENAPGS

SEQ ID NO: 256 - protein, 12 aa, artificial sequence
  skin binding peptide
KQATFPPNPTAY

SEQ ID NO: 257 - protein, 12 aa, artificial sequence
  skin binding peptide
HGHMVSTSQLSI

SEQ ID NO: 258 - protein, 7 aa, artificial sequence
  skin binding peptide
LSPSRMK

SEQ ID NO: 259 - protein, 7 aa, artificial sequence
  skin binding peptide
LPIPRMK

SEQ ID NO: 260 - protein, 7 aa, artificial sequence
  skin binding peptide
HQRPYLT

SEQ ID NO: 261 - protein, 7 aa, artificial sequence
  skin binding peptide
FPPLLRL

SEQ ID NO: 262 - protein, 12 aa, artificial sequence
  hair binding peptide
SVSVGMKPSPRP

SEQ ID NO: 263 - protein, 12 aa, artificial sequence
  hair binding peptide
LDVESYKGTSMP

SEQ ID NO: 264 - protein, 12 aa, artificial sequence
  hair binding peptide
RVPNKTVTVDGA

SEQ ID NO: 265 - protein, 12 aa, artificial sequence
  hair binding peptide
DRHKSKYSSTKS

SEQ ID NO: 266 - protein, 12 aa, artificial sequence
  hair binding peptide
KNFPQQKEFPLS

SEQ ID NO: 267 - protein, 12 aa, artificial sequence
  hair binding peptide
QRNSPPAMSRRD

SEQ ID NO: 268 - protein, 12 aa, artificial sequence
  hair binding peptide
TRKPNMPHGQYL

SEQ ID NO: 269 - protein, 12 aa, artificial sequence
  hair binding peptide
KPPHLAKLPFTT

SEQ ID NO: 270 - protein, 12 aa, artificial sequence
  hair binding peptide
NKRPPTSHRIHA

SEQ ID NO: 271 - protein, 12 aa, artificial sequence
  hair binding peptide
NLPRYQPPCKPL

SEQ ID NO: 272 - protein, 12 aa, artificial sequence
  hair binding peptide
RPPWKKPIPPSE

SEQ ID NO: 273 - protein, 12 aa, artificial sequence
  hair binding peptide
RQRPKDHFFSRP

SEQ ID NO: 274 - protein, 12 aa, artificial sequence
  Synthetic construct. hair binding peptide
SVPNKXVTVDGX

SEQ ID NO: 275 - protein, 12 aa, artificial sequence
  hair binding peptide
TTKWRHRAPVSP

SEQ ID NO: 276 - protein, 12 aa, artificial sequence
  hair binding peptide
WLGKNRIKPRAS

SEQ ID NO: 277 - protein, 12 aa, artificial sequence
  hair binding peptide
SNFKTPLPLTQS

SEQ ID NO: 278 - protein, 12 aa, artificial sequence
  hair binding peptide
KELQTRNVVQRE

SEQ ID NO: 279 - protein, 12 aa, artificial sequence
  hair binding peptide
GMPAMHWIHPFA

SEQ ID NO: 280 - protein, 12 aa, artificial sequence
  hair binding peptide
TPTANQFTQSVP

SEQ ID NO: 281 - protein, 12 aa, artificial sequence
  hair binding peptide
AAGLSQKHERNR

SEQ ID NO: 282 - protein, 12 aa, artificial sequence
  hair binding peptide
ETVHQTPLSDRP

SEQ ID NO: 283 - protein, 12 aa, artificial sequence
  hair binding peptide
LPALHIQRHPRM

SEQ ID NO: 284 - protein, 12 aa, artificial sequence
  hair binding peptide
QPSHSQSHNLRS

SEQ ID NO: 285 - protein, 12 aa, artificial sequence
  hair binding peptide
RGSQKSKPPRPP

SEQ ID NO: 286 - protein, 12 aa, artificial sequence
  hair binding peptide
THTQKTPLLYYH

SEQ ID NO: 287 - protein, 12 aa, artificial sequence
  hair binding peptide
TKGSSQAILKST

SEQ ID NO: 288 - protein, 7 aa, artificial sequence
  hair binding peptide
DLHTVYH

SEQ ID NO: 289 - protein, 7 aa, artificial sequence
  hair binding peptide
HIKPPTR

SEQ ID NO: 290 - protein, 7 aa, artificial sequence
  hair binding peptide
HPVWPAI

SEQ ID NO: 291 - protein, 7 aa, artificial sequence
  hair binding peptide
MPLYYLQ

SEQ ID NO: 292 - protein, 26 aa, artificial sequence
  hair binding peptide
HLTVPWRGGGSAVPFYSHSQITLPNH

SEQ ID NO: 293 - protein, 41 aa, artificial sequence
  hair binding peptide
GPHDTSSGGVRPNLHHTSKKEKRENRKVPFYSHSVTSRGNV

SEQ ID NO: 294 - protein, 7 aa, artificial sequence
  hair binding peptide
KHPTYRQ

SEQ ID NO: 295 - protein, 7 aa, artificial sequence
  hair binding peptide
HPMSAPR

SEQ ID NO: 296 - protein, 7 aa, artificial sequence
  hair binding peptide
MPKYYLQ

SEQ ID NO: 297 - protein, 7 aa, artificial sequence
  hair binding peptide
MHAHSIA

SEQ ID NO: 298 - protein, 7 aa, artificial sequence
  hair binding peptide
TAATTSP

SEQ ID NO: 299 - protein, 7 aa, artificial sequence
  hair binding peptide
LGIPQNL

SEQ ID NO: 300 - protein, 12 aa, artificial sequence
  hair binding peptide
AKPISQHLQRGS

SEQ ID NO: 301 - protein, 12 aa, artificial sequence
  hair binding peptide
APPTPAAASATT

SEQ ID NO: 302 - protein, 12 aa, artificial sequence
  hair binding peptide
DPTEGARRTIMT

SEQ ID NO: 303 - protein, 12 aa, artificial sequence
  hair binding peptide
EQISGSLVAAPW

SEQ ID NO: 304 - protein, 12 aa, artificial sequence
  hair binding peptide
LDTSFPPVPFHA

SEQ ID NO: 305 - protein, 11 aa, artificial sequence
  hair binding peptide
LPRIANTWSPS

SEQ ID NO: 306 - protein, 12 aa, artificial sequence
  hair binding peptide
RTNAADHPAAVT

SEQ ID NO: 307 - protein, 12 aa, artificial sequence
  hair binding peptide
SLNWVTIPGPKI

SEQ ID NO: 308 - protein, 12 aa, artificial sequence
  hair binding peptide
TDMQAPTKSYSN

SEQ ID NO: 309 - protein, 12 aa, artificial sequence
  hair binding peptide
TIMTKSPSLSCG

SEQ ID NO: 310 - protein, 12 aa, artificial sequence
  hair binding peptide
TPALDGLRQPLR

SEQ ID NO: 311 - protein, 12 aa, artificial sequence
  hair binding peptide
TYPASRLPLLAP

SEQ ID NO: 312 - protein, 12 aa, artificial sequence
  hair binding peptide
AKTHKHPAPSYS

SEQ ID NO: 313 - protein, 12 aa, artificial sequence
  hair binding peptide
TDPTPFSISPER

SEQ ID NO: 314 - protein, 20 aa, artificial sequence
  hair binding peptide
CAAGCCTCAGCGACCGAATA

SEQ ID NO: 315 - protein, 12 aa, artificial sequence
  hair binding peptide
WHDKPQNSSKST

SEQ ID NO: 316 - protein, 12 aa, artificial sequence
  hair binding peptide
NEVPARNAPWLV

SEQ ID NO: 317 - protein, 13 aa, artificial sequence
  hair binding peptide
NSPGYQADSVAIG

SEQ ID NO: 318 - protein, 12 aa, artificial sequence
  hair binding peptide
TQDSAQKSPSPL

SEQ ID NO: 319 - protein, 12 aa, artificial sequence
  hair binding peptide
TPPELLHGDPRS

SEQ ID NO: 320 - protein, 12 aa, artificial sequence
  hair binding peptide
TPPTNVLMLATK

SEQ ID NO: 321 - protein, 7 aa, artificial sequence
  hair binding peptide
NTSQLST

SEQ ID NO: 322 - protein, 7 aa, artificial sequence
  hair binding peptide
NTPKENW

SEQ ID NO: 323 - protein, 7 aa, artificial sequence
  hair binding peptide
NTPASNR

SEQ ID NO: 324 - protein, 7 aa, artificial sequence
  hair binding peptide
PRGMLST

SEQ ID NO: 325 - protein, 7 aa, artificial sequence
  hair binding peptide
PPTYLST

SEQ ID NO: 326 - protein, 12 aa, artificial sequence
  hair binding peptide
TIPTHRQHDYRS

SEQ ID NO: 327 - protein, 7 aa, artificial sequence
  hair binding peptide
TPPTHRL

SEQ ID NO: 328 - protein, 7 aa, artificial sequence
  hair binding peptide
LPTMSTP

SEQ ID NO: 329 - protein, 7 aa, artificial sequence
  hair binding peptide
LGTNSTP

SEQ ID NO: 330 - protein, 12 aa, artificial sequence
  hair binding peptide
TPLTGSTNLLSS

SEQ ID NO: 331 - protein, 7 aa, artificial sequence
  hair binding peptide
TPLTKET

SEQ ID NO: 332 - protein, 7 aa, artificial sequence
  hair binding peptide
QQSHNPP

SEQ ID NO: 333 - protein, 7 aa, artificial sequence
  hair binding peptide
TQPHNPP

SEQ ID NO: 334 - protein, 12 aa, artificial sequence
  hair binding peptide
STNLLRTSTVHP

SEQ ID NO: 335 - protein, 12 aa, artificial sequence
  hair binding peptide
HTQPSYSSTNLF

SEQ ID NO: 336 - protein, 7 aa, artificial sequence
  hair binding peptide
SLLSSHA

SEQ ID NO: 337 - protein, 12 aa, artificial sequence
  hair binding peptide
QQSSISLSSHAV

SEQ ID NO: 338 - protein, 7 aa, artificial sequence
  hair binding peptide
NASPSSL

SEQ ID NO: 339 - protein, 7 aa, artificial sequence
  hair binding peptide
HSPSSLR

SEQ ID NO: 340 - protein, 7 aa, artificial sequence
  Synthetic construct. hair binding peptide
KXSHHTH

SEQ ID NO: 341 - protein, 7 aa, artificial sequence
  Synthetic construct. hair binding peptide
EXSHHTH

SEQ ID NO: 342 - protein, 7 aa, artificial sequence
  hair binding peptide
LESTSLL

SEQ ID NO: 343 - protein, 7 aa, artificial sequence
  hair binding peptide
TPLTKET

SEQ ID NO: 344 - protein, 7 aa, artificial sequence
  hair binding peptide
KQSHNPP

SEQ ID NO: 345 - protein, 9 aa, artificial sequence
  hair binding peptide
STLHKYKSQ

SEQ ID NO: 346 - protein, 12 aa, artificial sequence
  hair binding peptide
YPSFSPTYRPAF

SEQ ID NO: 347 - protein, 12 aa, artificial sequence
  hair binding peptide
ALPRIANTWSPS

SEQ ID NO: 348 - protein, 8 aa, artificial sequence
  hair binding peptide
LESTPKMK

SEQ ID NO: 349 - protein, 67 aa, artificial sequence
  hair binding peptide
PNTSQLSTGGGRTNAADHPKCGGGNTSQLSTGGGRTNAADHPKCGGGNTSQLSTGGGRTNAADHPKC

SEQ ID NO: 350 - protein, 56 aa, artificial sequence
  hair binding peptide
DPRTNAADHPAAVTGGGCGGGRTNAADHPAAVTGGGCGGGRTNAADHPAAVTGGGC

SEQ ID NO: 351 - protein, 51 aa, artificial sequence
  hair binding peptide
DPRTNAADHPAAVTGGGCGGGIPWWNIRAPLNAGGGCGGGDLTLPFHGGGC

SEQ ID NO: 352 - protein, 58 aa, artificial sequence
  hair binding peptide
RTNAADHPAAVTGGGCDPGGGRTNAADHPAAVTGGGCDPGGGRTNAADHPAAVTGGGC

SEQ ID NO: 353 - protein, 83 aa, artificial sequence
  hair binding peptide
DPTPPTNVLMLATKGGGRTNAADHPKCGGGTPPTNVLMLATKGGGRTNAADHPKCGGGTPPTNVLMLATKGGGRTNAADHPKC

SEQ ID NO: 354 - protein, 83 aa, artificial sequence
  hair binding peptide
DPRTNAADHPGGGTPPTNVLMLATKKCGGGRTNAADHPGGGTPPTNVLMLATKKCGGGRTNAADHPGGGTPPTNVLMLATKKC

SEQ ID NO: 355 - protein, 12 aa, artificial sequence
  Nail binding peptide
ALPRIANTWSPS

SEQ ID NO: 356 - protein, 12 aa, artificial sequence
  Nail binding peptide
YPSFSPTYRPAF

SEQ ID NO: 357 - protein, 17 aa, artificial sequence
  antimicrobial peptide
PKGLKKLLKGLKKLLKL

SEQ ID NO: 358 - protein, 16 aa, artificial sequence
  antimicrobial peptide
KGLKKLLKGLKKLLKL

SEQ ID NO: 359 - protein, 16 aa, artificial sequence
  antimicrobial peptide
KGLKKLLKLLKKLLKL

SEQ ID NO: 360 - protein, 14 aa, artificial sequence
  antimicrobial peptide
LKKLLKLLKKLLKL

SEQ ID NO: 361 - protein, 12 aa, artificial sequence
  antimicrobial peptide
LKKLLKLLKKLL

SEQ ID NO: 362 - protein, 17 aa, artificial sequence
  antimicrobial peptide
VAKKLAKLAKKLAKLAL

SEQ ID NO: 363 - protein, 13 aa, artificial sequence
  antimicrobial peptide
FAKLLAKALKKLL

SEQ ID NO: 364 - protein, 16 aa, artificial sequence
  antimicrobial peptide
KGLKKGLKLLKKLLKL

SEQ ID NO: 365 - protein, 16 aa, artificial sequence
  antimicrobial peptide
KGLKKLLKLGKKLLKL

SEQ ID NO: 366 - protein, 16 aa, artificial sequence
  antimicrobial peptide
KGLKKLGKLLKKLLKL

SEQ ID NO: 367 - protein, 16 aa, artificial sequence
  antimicrobial peptide
KGLKKLLKLLKKGLKL

SEQ ID NO: 368 - protein, 16 aa, artificial sequence
  antimicrobial peptide
KGLKKLLKLLKKLGKL

SEQ ID NO: 369 - protein, 19 aa, artificial sequence
  antimicrobial peptide
FALALKALKKLKKALKKAL

SEQ ID NO: 370 - protein, 17 aa, artificial sequence
  antimicrobial peptide
FAKKLAKLAKKLAKLAL

SEQ ID NO: 371 - protein, 13 aa, artificial sequence
  antimicrobial peptide
FAKLLAKLAKKLL

SEQ ID NO: 372 - protein, 15 aa, artificial sequence
  antimicrobial peptide
FAKKLAKLALKLAKL

SEQ ID NO: 373 - protein, 10 aa, artificial sequence
  antimicrobial peptide
FAKKLAKKLL

SEQ ID NO: 374 - protein, 13 aa, artificial sequence
  antimicrobial peptide
FAKLLAKLAKKVL

SEQ ID NO: 375 - protein, 13 aa, artificial sequence
  antimicrobial peptide
KYKKALKKLAKLL

SEQ ID NO: 376 - protein, 12 aa, artificial sequence
  antimicrobial peptide
FALLKALLKKAL

SEQ ID NO: 377 - protein, 14 aa, artificial sequence
  antimicrobial peptide
KRLFKKLKFSLRKY

SEQ ID NO: 378 - protein, 14 aa, artificial sequence
  antimicrobial peptide
KRLFKKLLFSLRKY

SEQ ID NO: 379 - protein, 14 aa, artificial sequence
  antimicrobial peptide
LLLFLLKKRKKRKY

SEQ ID NO: 380 - protein, 36 aa, Hyalophora cecropia
KWKLFKKIEKVGQNIRDGIIKAGPAVAWGQATQIAK

SEQ ID NO: 381 - protein, 23 aa, Xenopus sp.
GIGKFLHSAKKFGKAFVGEIMNS

SEQ ID NO: 382 - protein, 22 aa, Xenopus sp.
GIGKFLKKAKKFGKAFVKILKK

SEQ ID NO: 383 - protein, 12 aa, Bos Taurus
RLCRIVVIRVCR

SEQ ID NO: 384 - protein, 13 aa, Bos sp.
ILPWKWPWWPWRR

SEQ ID NO: 385 - protein, 24 aa, Homo sapiens
DSHAKRHHGYKRKFHEKHHSHRGY

SEQ ID NO: 386 - protein, 7 aa, artificial sequence
  pigment binding peptide
MPPPLMQ

SEQ ID NO: 387 - protein, 7 aa, artificial sequence
  pigment binding peptide
FHENWPS

SEQ ID NO: 388 - protein, 12 aa, artificial sequence
  pigment binding peptide
RTAPTTPLLLSL

SEQ ID NO: 389 - protein, 12 aa, artificial sequence
  pigment binding peptide
WHLSWSPVPLPT

SEQ ID NO: 390 - protein, 7 aa, artificial sequence
  pigment binding peptide
PHARLVG

SEQ ID NO: 391 - protein, 7 aa, artificial sequence
  pigment binding peptide
NIPYHHP

SEQ ID NO: 392 - protein, 7 aa, artificial sequence
  pigment binding peptide
TTMPAIP

SEQ ID NO: 393 - protein, 7 aa, artificial sequence
  pigment binding peptide
HNLPPRS

SEQ ID NO: 394 - protein, 12 aa, artificial sequence
  pigment binding peptide
AHKTQMGVRQPA

SEQ ID NO: 395 - protein, 12 aa, artificial sequence
  pigment binding peptide
ADNVQMGVSHTP

SEQ ID NO: 396 - protein, 12 aa, artificial sequence
  pigment binding peptide
AHNAQMGVSHPP

SEQ ID NO: 397 - protein, 12 aa, artificial sequence
  pigment binding peptide
ADYVGMGVSHRP

SEQ ID NO: 398 - protein, 12 aa, artificial sequence
  pigment binding peptide
SVSVGMKPSPRP

SEQ ID NO: 399 - protein, 7 aa, artificial sequence
  pigment binding peptide
YPNTALV

SEQ ID NO: 400 - protein, 7 aa, artificial sequence
  pigment binding peptide
VATRIVS

SEQ ID NO: 401 - protein, 12 aa, artificial sequence
  pigment binding peptide
HSLKNSMLTVMA

SEQ ID NO: 402 - protein, 7 aa, artificial sequence
  pigment binding peptide
NYPTQAP

SEQ ID NO: 403 - protein, 7 aa, artificial sequence
  pigment binding peptide
KCCYSVG

SEQ ID NO: 404 - protein, 12 aa, artificial sequence
  pigment binding peptide
RHDLNTWLPPVK

SEQ ID NO: 405 - protein, 12 aa, artificial sequence
  pigment binding peptide
EISLPAKLPSAS

SEQ ID NO: 406 - protein, 12 aa, artificial sequence
  pigment binding peptide
SVSVGMKPSPRP

SEQ ID NO: 407 - protein, 12 aa, artificial sequence
  pigment binding peptide
SDYVGMRPSPRH

SEQ ID NO: 408 - protein, 12 aa, artificial sequence
  pigment binding peptide
SDYVGMRLSPSQ

SEQ ID NO: 409 - protein, 12 aa, artificial sequence
  pigment binding peptide
SVSVGIQPSPRP

SEQ ID NO: 410 - protein, 12 aa, artificial sequence
  pigment binding peptide
YVSVGIKPSPRP

SEQ ID NO: 411 - protein, 12 aa, artificial sequence
  pigment binding peptide
YVCEGIHPCPRP

SEQ ID NO: 412 - protein, 7 aa, artificial sequence
  cellulose binding peptide
VPRVTSI

SEQ ID NO: 413 - protein, 7 aa, artificial sequence
  cellulose binding peptide
MANHNLS

SEQ ID NO: 414 - protein, 7 aa, artificial sequence
  cellulose binding peptide
FHENWPS

SEQ ID NO: 415 - protein, 12 aa, artificial sequence
  cellulose binding peptide
THKTSTQRLLAA

SEQ ID NO: 416 - protein, 12 aa, artificial sequence
  cellulose binding peptide
KCCYVNVGSVFS

SEQ ID NO: 417 - protein, 12 aa, artificial sequence
  cellulose binding peptide
AHMQFRTSLTPH

SEQ ID NO: 418 - protein, 12 aa, artificial sequence
  PET binding peptide
GTSDHMIMPFFN

SEQ ID NO: 419 - protein, 12 aa, artificial sequence
  PMMA binding peptide
IPWWNIRAPLNA

SEQ ID NO: 420 - protein, 12 aa, artificial sequence
  PMMA binding peptide
TAVMNVVNNQLS

SEQ ID NO: 421 - protein, 12 aa, artificial sequence
  PMMA binding peptide
VPWWAPSKLSMQ

SEQ ID NO: 422 - protein, 12 aa, artificial sequence
  PMMA binding peptide
MVMAPHTPRARS

SEQ ID NO: 423 - protein, 12 aa, artificial sequence
  PMMA binding peptide
TYPNWAHLLSHY

SEQ ID NO: 424 - protein, 7 aa, artificial sequence
  PMMA binding peptide
TPWWRIT

SEQ ID NO: 425 - protein, 7 aa, artificial sequence
  PMMA binding peptide
DLTLPFH

SEQ ID NO: 426 - protein, 7 aa, artificial sequence
  PMMA binding peptide
GTSIPAM

SEQ ID NO: 427 - protein, 7 aa, artificial sequence
  PMMA binding peptide
HHKHVVA

SEQ ID NO: 428 - protein, 7 aa, artificial sequence
  PMMA binding peptide
HHHKHFM

SEQ ID NO: 429 - protein, 7 aa, artificial sequence
  PMMA binding peptide
HHHRHQG

SEQ ID NO: 430 - protein, 7 aa, artificial sequence
  PMMA binding peptide
HHWHAPR

SEQ ID NO: 431 - protein, 7 aa, artificial sequence
  Nylon binding peptide
KTPPTRP

SEQ ID NO: 432 - protein, 7 aa, artificial sequence
  Nylon binding peptide
VINPNLD

SEQ ID NO: 433 - protein, 7 aa, artificial sequence
  Nylon binding peptide
KVWIVST

SEQ ID NO: 434 - protein, 7 aa, artificial sequence
  Nylon binding peptide
AEPVAML

SEQ ID NO: 435 - protein, 7 aa, artificial sequence
  Nylon binding peptide
AELVAML

SEQ ID NO: 436 - protein, 7 aa, artificial sequence
  Nylon binding peptide
HSLRLDW

SEQ ID NO: 437 - protein, 12 aa, artificial sequence
  PTFE binding peptide
ESSYSWSPARLS

SEQ ID NO: 438 - protein, 12 aa, artificial sequence
  PTFE binding peptide
GPLKLLHAWWQP

SEQ ID NO: 439 - protein, 7 aa, artificial sequence
  PTFE binding peptide
NALTRPV

SEQ ID NO: 440 - protein, 7 aa, artificial sequence
  PTFE binding peptide
SAPSSKN

SEQ ID NO: 441 - protein, 12 aa, artificial sequence
  PTFE binding peptide
SVSVGMKPSPRP

SEQ ID NO: 442 - protein, 12 aa, artificial sequence
  PTFE binding peptide
SYYSLPPIFHIP

SEQ ID NO: 443 - protein, 12 aa, artificial sequence
  PTFE binding peptide
TFTPYSITHALL

SEQ ID NO: 444 - protein, 12 aa, artificial sequence
  PTFE binding peptide
TMGFTAPRFPHY

SEQ ID NO: 445 - protein, 12 aa, artificial sequence
  PTFE binding peptide
TNPFPPPPSSPA

SEQ ID NO: 446 - protein, 5 aa, artificial sequence
  Synthetic construct.  Caspase-3 cleavage sequence
DMQDX

(19) United States
(12) Patent Application Publication
(10) Pub. No.: US 2010/0234568 A1
(43) Pub. Date: Sep. 16, 2010

(54) Identification of peptide tags for the production of insoluble peptides by sequence scanning

(76) Inventors: Linda Jane Decarolis, Wilmington, DE (US); Stephen R. Fahnestock, Wilmington, DE (US); Pierre E. Rouviere, Wilmington, DE (US); Hong Wang, Kennett Square, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/641,981

(22) Filed: Dec. 19, 2006

Related U.S. Application Data
(60) Provisional application No. 60/852,841, filed on Oct. 19, 2006.

(51) Int. Cl. C07K 14/00; C40B 30/04

(52) U.S. Cl. 530/350; 506/9

(57) ABSTRACT

A method is provided to identify short peptide tags, referred to here as inclusion body tags (IBTs), useful for the generation of insoluble fusion peptides. A library of genetic constructs were prepared encoding fusion peptides comprising an inclusion body tag of 10-50 contiguous amino acids from a full-length insoluble protein operably linked to a peptide of interest. The library was designed to include a sufficient number of overlapping inclusion body tags to ensure that the entire length of the full-length insoluble protein was represented. Host cells transformed and expressing the genetic constructs were evaluated for inclusion body formation.

This application claims priority under 35 U.S.C. §119 from U.S. Provisional Application Ser. No. 60/852,841, filed Oct. 19, 2006.

### FIELD OF THE INVENTION

The invention relates to the field of protein expression from microbial cells. More specifically, a method to identify short peptide tags useful in the preparation of insoluble fusion peptides is provided.

### BACKGROUND OF THE INVENTION

The efficient production of bioactive proteins and peptides has become a hallmark of the biomedical and industrial biochemical industry. Bioactive peptides and proteins are used as curative agents in a variety of diseases such as diabetes (insulin), viral infections and leukemia (interferon), diseases of the immune system (interleukins), and red blood cell deficiencies (erythropoietin) to name a few. Additionally, large quantities of proteins and peptides are needed for various industrial applications including, for example, the pulp and paper and pulp industries, textiles, food industries, sugar refining, wastewater treatment, production of alcoholic beverages and as catalysts for the generation of new pharmaceuticals.

With the advent of the discovery and implementation of combinatorial peptide screening technologies such as bacterial display (Kemp, D. J.; Proc. Natl. Acad. Sci. USA 78(7): 4520-4524 (1981); yeast display (Chien et al., Proc Natl Acad Sci USA 88(21): 9578-82 (1991)), combinatorial solid phase peptide synthesis (U.S. Pat. No. 5,449,754, U.S. Pat. No. 5,480,971, U.S. Pat. No. 5,585,275, U.S. Pat. No. 5,639,603), and phage display technology (U.S. Pat. No. 5,223,409, U.S. Pat. No. 5,403,484, U.S. Pat. No. 5,571,698, U.S. Pat. No. 5,837,500) new applications for peptides having specific binding affinities have been developed. In particular, peptides are being looked to as linkers in biomedical fields for the attachment of diagnostic and pharmaceutical agents to surfaces (see Grinstaff et al, U.S. Patent Application Publication No. 2003/0185870 and Linter in U.S. Pat. No. 6,620,419), as well as in the personal care industry for the attachment of benefit agents to body surfaces such as hair and skin (see commonly owned U.S. patent application Ser. No. 10/935,642, and Janssen et al. U.S. Patent Application Publication No. 2003/0152976), and in the printing industry for the attachment of pigments to print media (see commonly owned U.S. patent application Ser. No. 10/935,254).

In some limited situations, commercially useful proteins and peptides may be synthetically generated or isolated from natural sources. However, these methods are often expensive, time consuming and characterized by limited production capacity. The preferred method of protein and peptide production is through the fermentation of recombinantly constructed organisms, engineered to over-express the protein or peptide of interest. Although preferable to synthesis or isolation, recombinant expression of peptides has a number of obstacles to be overcome in order to be a cost-effective means of production. For example, peptides (and in particular short peptides) produced in a cellular environment are susceptible to degradation from the action of native cellular proteases. Additionally, purification can be difficult, resulting in poor yields depending on the nature of the protein or peptide of interest.

One means to mitigate the above difficulties is the use the genetic chimera for protein and peptide expression. A chimeric protein or “fusion protein” is a polypeptide comprising at least one portion of the desired protein product fused to at least one portion comprising a peptide tag. The peptide tag may be used to assist protein folding, assist post expression purification, protect the protein from the action of degradative enzymes, and/or assist the protein in passing through the cell membrane.

In many cases it is useful to express a protein or peptide in insoluble form, particularly when the peptide of interest is rather short, normally soluble, and subject to proteolytic degradation within the host cell. Production of the peptide in insoluble form both facilitates simple recovery and protects the peptide from the undesirable proteolytic degradation. One means to produce the peptide in insoluble form is to recombinantly produce the peptide as part of an insoluble fusion protein by including in the fusion construct at least one peptide tag (i.e., an inclusion body tag) that induces inclusion body formation. Typically, the fusion protein is designed to include at least one cleavable peptide linker so that the peptide of interest can be subsequently recovered from the fusion protein. The fusion protein may be designed to include a plurality of inclusion body tags, cleavable peptide linkers, and regions encoding the peptide of interest.

Fusion proteins comprising a carrier protein tag that facilitates the expression of insoluble proteins are well known in the art. Typically, the tag portion of the chimeric or fusion protein is large, increasing the likelihood that the fusion protein will be insoluble. Example of large peptide tags typically used include, but are not limited to chloramphenicol acetyltransferase (Dykes et al., Eur. J. Biochem., 174:411 (1988), β-galactosidase (Schellenberger et al., Int. J. Peptide Protein Res., 41:326′ (1993); Shen et al., Proc. Nat. Acad. Sci. USA 281:4627 (1984); and Kempe et al., Gene, 39:239 (1985)), glutathione-S-transferase (Ray et al., Bio/Technology, 11:64 (1993) and Hancock et al. (WO94/04688)), the N-terminus of L-ribulokinase (U.S. Pat. No. 5,206,154 and Lai et al., Antimicrob. Agents & Chemo., 37:1614 (1993), bacteriophage T4 gp55 protein (Gramm et al., Bio/Technology, 12:1017 (1994), bacterial ketosteroid isomerase protein (Kuliopulos et al., J. Am. Chem. Soc. 116:4599 (1994), ubiquitin (Pilon et al., Biotechnol. Prog., 13:374-79 (1997), bovine prochymosin (Haught et al., Biotechnol. Bioengineer. 57:55-61 (1998), and bactericidal/permeability-increasing protein (“BPI”; Better, M. D. and Gavit, P D., U.S. Pat. No. 6,242,219). The art is replete with specific examples of this technology, see for example U.S. Pat. No. 6,613,548, describing fusion protein of proteinaceous tag and a soluble protein and subsequent purification from cell lysate; U.S. Pat. No. 6,037,145, teaching a tag that protects the expressed chimeric protein from a specific protease; U.S. Pat. No. 5,648,244, teaching the synthesis of a fusion protein having a tag and a cleavable linker for facile purification of the desired protein; and U.S. Pat. No. 5,215,896; U.S. Pat. No. 5,302,526; U.S. Pat. No. 5,330,902; and US 2005221444, describing fusion tags containing amino acid compositions specifically designed to increase insolubility of the chimeric protein or peptide.

Recombinant production of a short peptide using a large, insoluble carrier protein decreases the production efficiency of the desired peptide it is only makes up a small percentage of the total mass of the purified fusion protein. This is particularly problematic in situations where the desired protein or peptide is small. In such situations it is advantageous to use a small fusion tags (i.e., short peptides capable of inducing inclusion body formation, herein referred to as “inclusion body tags”) to maximized yield.

Limited numbers of effective, short, inclusion body tags have been reported in the art. Their effectiveness may depend on the peptide targeted for production. The identification of suitable short peptide tags often relies, to a great extent, on serendipity. As such, a method to identify short peptide tags having the ability to induce the formation insoluble fusion protein is needed.

Many of the carrier proteins used in the art were selected base on previous observations about their inherent insolubility. However, their insolubility may be attributed to small portions of the total protein. The structure of these small regions responsible for inducing insoluble fusion protein formation is somewhat unpredictable. As such, an efficient method to identify small regions within the larger insoluble protein is need.

The problem to be solved is to provide a simple and efficient method to identify short peptides that facilitate insoluble fusion protein formation when operably linked to a short peptide-of-interest.

### SUMMARY OF THE INVENTION

A method is provided for identifying short peptides (inclusion body tags) that are useful for synthesizing insoluble fusion proteins. Short inclusion body tags are particularly useful for increasing expression and simplifying purification of short peptides (“peptides of interest”), especially short peptides useful in affinity applications.

The present method identifies short peptide tags (typically less than 50 amino acid in length) that are useful as inclusion body tags from a large insoluble protein or a protein having significant amino acid sequence homology to large insoluble protein.

Accordingly, a method to identify an inclusion body tag from a large insoluble protein is provided comprising:
a) providing a first genetic construct encoding an insoluble full-length protein;
b) constructing a first library of nucleic acid fragments from the first genetic construct of (a), each fragment encoding an inclusion body peptide tag of about 10-50 amino acids such that the peptide tags are generated beginning at the N-terminal region of the peptide and extending to the C-terminal end of the peptide, each peptide tag overlapping with the next peptide tag by about 3 to about 10 amino acids;
c) providing a second genetic construct encoding a target peptide to be expressed in insoluble form;
d) constructing a second library by combining, in combinatorial fashion, the nucleic acid fragments of the first library and the second genetic construct encoding the target peptide to create a library of expressible chimeric constructs; wherein each expressible chimeric construct within the library of expressible chimeric constructs encodes a fusion peptide;
e) transforming host cells with the library of expressible chimeric constructs of (d);
f) growing the transformed host cells of (e) under conditions wherein each expressible chimeric constructs is expressed as said fusion peptide
g) selecting the transformed host cells comprising said fusion peptide expressed in insoluble form;
h) identifying the inclusion body tag from the insoluble fusion peptide of (g); and
i) optionally isolating the identified inclusion body tag.

In another embodiment, the present invention provides an inclusion body tag identified by the above process.

### BRIEF DESCRIPTION OF THE BIOLOGICAL SEQUENCES

The following sequences comply with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPC and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

A Sequence Listing is provided herewith on Compact Disk. The contents of the Compact Disk containing the Sequence Listing are hereby incorporated by reference in compliance with 37 CFR 1.52(e). The Compact Disks are submitted in triplicate and are identical to one another. The disks are labeled “Copy 1—Sequence Listing”, “Copy 2—Sequence Listing”, and CRF. The disks contain the following file: CL3005 US NA.ST25 having the following size: 118,000 bytes and which was created Nov. 30, 2006.

SEQ ID NO: 1 is the nucleotide sequence of the TBP1 coding sequence encoding the TBP101 peptide.

SEQ ID NO: 2 is the amino acid sequence of the TBP101 peptide.

SEQ ID NOs: 3-7 are the nucleotide sequences of oligonucleotides used to synthesize TBP1.

SEQ ID NO: 8 and 9 are the nucleotide sequences of the primers used to PCR amplify TBP1.

SEQ ID NO: 10 is the nucleotide sequence of pENTRT™/D-TOPO® plasmid (Invitrogen, Carlsbad, Calif.).

SEQ ID NO: 11 is the nucleotide sequence of the pDEST plasmid (Invitrogen).

SEQ ID NO: 12 is the nucleotide sequence of the coding region encoding the INK101 fusion peptide.

SEQ ID NO: 13 is the amino acid sequence of the INK101 fusion peptide.

SEQ ID NO: 14 is the nucleotide sequence of plasmid pLX121.

SEQ ID NOs: 15 and 16 are the nucleotide sequences of primers used to introduce an acid cleavable aspartic acid-proline dipeptide linker into TBP101.

SEQ ID NO: 17 is the nucleotide sequence of the coding region encoding the INK101DP peptide.

SEQ ID NO: 18 is the amino acid sequence of the INK101DP peptide.

SEQ ID NO: 19 is the nucleotide sequence of the opaque2 modifier (referred to herein as “gamma zeinA”) coding region from Zea mays.

SEQ ID NO: 20 is the amino acid sequence of the 27-kDa gamma zeinA protein (GenBank® AAP32017).

SEQ ID NOs: 21 to 110 are the nucleotide sequences of oligonucleotides used to prepare the zein-based inclusion body tags.

SEQ ID NOs: 111 to 155 and 157 to 158 are the amino acid sequences of zein-based peptides evaluated as potential inclusion body tags.

SEQ ID NO: 156 is the amino acid sequence of the T7 translation enhancer element found in IBT-180 and IBT-181.

SEQ ID NO: 159 is the nucleotide sequence of the coding region for the gene encoding the Daucus carota (carrot) extracellular cystatin protein (GenBank® BAA20464).

SEQ ID NO: 160 is the amino acid sequence of the Daucus carota extracellular cystatin protein (GenBank® BAA20464).

SEQ ID NOs: 161 to 222 are the nucleotide sequences of oligonucleotides used to prepare the cystatin-based inclusion body tags.

SEQ ID NOs: 223 to 253 are the amino acid sequences of the cystatin-based peptides evaluated as potential inclusion body tags.

SEQ ID NOs: 254 to 356 are examples of amino acid sequences of body surface binding peptides, SEQ ID NOs 254-261 are skin binding peptides, SEQ ID NOs 262-354 are hair binding peptides, and SEQ ID NOs: 355-356 are nail binding peptides.

SEQ ID NOs: 356 to 385 are examples of antimicrobial peptide sequences.

SEQ ID NOs: 386 to 411 are examples of pigment binding peptides,

SEQ ID NOs: 386-389 bind carbon black, SEQ ID NOs: 390-398 are Cromophtal® yellow (Ciba Specialty Chemicals, Basel, Switzerland) binding peptides, SEQ ID NOs: 399-401 are Sunfast® magenta (Sun Chemical Corp., Parsippany, N.J.) binding peptides, and SEQ ID NOs: 402-411 are Sunfast® blue binding peptides.

SEQ ID NOs: 412 to 445 are examples of polymer binding peptides, SEQ ID NOs: 412-417 are cellulose binding peptides, SEQ ID NO: 418 is a polyethylene terephthalate) (PET) binding peptide, SEQ ID NOs: 419-430 are poly(methyl methacrylate) (PMMA) binding peptides, SEQ ID NOs: 431-436 are nylon binding peptides, and SEQ ID NOs: 437-445 are poly(tetrafluoro ethylene) (PTFE) binding peptides.

SEQ ID NO: 446 is the amino acid sequence of the Caspase-3 cleavage site that may be used as a cleavable peptide linker domain.

### DETAILED DESCRIPTION OF THE INVENTION

The present invention provides a method to identify short peptide tags (“inclusion body tag fusion partners”) derived from a larger insoluble protein that may be coupled with a peptide of interest to form an insoluble fusion protein. In this manner, short inclusion body tags can be identified quickly and efficiently.

Specifically, a library of chimeric genes encoding fusion proteins was designed to assess the ability of small peptide tags derived from a larger, insoluble protein to induce the formation of insoluble inclusion bodies when fused to a short, soluble peptide of interest. A library of peptide tags comprising 10 to 50 contiguous amino acids from a larger, insoluble protein was prepared such that the peptide tags were generated beginning at the N-terminal region of the insoluble full length protein and extending to the C-terminal end of the insoluble full length protein, each peptide tag overlapping with the next peptide tag by about 3 to about 10 amino acids. In this way, the larger, insoluble protein was “scanned” or “probed” for small regions suitable for use as potential inclusion body tags in a method referred to herein as “tag scanning” or “sequence scanning”.

The present method provides a means to identify short inclusion body tags useful for the expression and recovery of short peptides of interest. Such peptides typically have high value in any number of applications including, but not limited to medical, biomedical, diagnostic, personal care, and affinity applications where the peptides of interest are used as linkers to various surfaces.

The following definitions are used herein and should be referred to for interpretation of the claims and the specification. Unless otherwise noted, all U.S. patents and U.S. patent applications referenced herein are incorporated by reference in their entirety.

As used herein, the term “comprising” means the presence of the stated features, integers, steps, or components as referred to in the claims, but that it does not preclude the presence or addition of one or more other features, integers, steps, components or groups thereof.

The term “invention” or “present invention” as used herein is a non-limiting term and is not intended to refer to any single embodiment of the particular invention but encompasses all possible embodiments as described in the specification and the claims.

“Open reading frame” is abbreviated ORF.

“Polymerase chain reaction” is abbreviated PCR.

As used herein, the term “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

As used herein, the term “hair” as used herein refers to human hair, eyebrows, and eyelashes.

As used herein, the term “skin” as used herein refers to human skin, or substitutes for human skin, such as pig skin, Vitro-Skin® and EpiDerm™. Skin, as used herein, will refer to a body surface generally comprising a layer of epithelial cells and may additionally comprise a layer of endothelial cells.

As used herein, the term “nails” as used herein refers to human fingernails and toenails and other body surfaces comprising primarily keratin.

As used herein, the term “pigment” refers to an insoluble, organic or inorganic colorant.

As used herein, “HBP” means hair-binding peptide. Examples of hair binding peptides have been reported (U.S. patent application Ser. No. 11/074,473 to Huang et al.; WO 0179479; U.S. Patent Application Publication No. 2002/0098524 to Murray et al.; Janssen et al., U.S. Patent Application Publication No. 2003/0152976 to Janssen et al.; WO 04048399; U.S. Provisional Patent Application No. 60/721,329; U.S. Provisional Application No. 60/721,329, and U.S. Provisional Patent Application No. 60/790,149).

As used herein, “SBP” means skin-binding peptide. Examples of skin binding peptides have also been reported (U.S. patent application Ser. No. 11/069,858 to Buseman-Williams; Rothe et. al., WO 2004/000257; and U.S. Provisional Patent Application No. 60/790,149).

As used herein, “NBP” means nail-binding peptide. Examples of nail binding peptides have been reported (U.S. Provisional Patent Application No. 60/790,149).

As used herein, an “antimicrobial peptide” is a peptide having the ability to kill microbial cell populations (U.S. Provisional Patent Application No. 60/790,149).

As used herein, the terms “cystatin”, “cystatin protein”, “Daucus carota cystatin”, and “extracellular insoluble cystatin” will refer to the Daucus carota protein having the amino acid sequence as set forth in SEQ ID NO: 160 (GenBank® Accession No. BAA20464). The coding region of the cystatin gene having GenBank® Accession No. BAA20464 is provided as SEQ ID NO: 159. As used herein, “cystatin-based” inclusion body tags are short peptides derived from a portion of the cystatin protein (SEQ ID NO: 160).

As used herein, the terms “zein 27 kDa storage protein”, “zein protein”, “gamma zein protein”, and “opaque2 protein” will refer to the Zea mays protein having the amino acid sequence as set forth in SEQ ID NO:20 (GenBank® Accession No. AAP32017). The coding region encoding the zein protein having GenBank® Accession No. AAP32017 is provided as SEQ ID NO: 19. As used herein, “zein-based” inclusion body tags are short peptides derived from a portion of the Zea mays zein protein as set forth in SEQ ID NO: 20.

As used herein, the term “inclusion body tag” will be abbreviated “IBT” and will refer a polypeptide that facilitates/stimulates formation of inclusion bodies when fused to a peptide of interest. The peptide of interest is typically short and soluble within the host cell and/or host cell lysate when not fused to an inclusion body tag. Fusion of the peptide of interest to the inclusion body tag produces an insoluble fusion protein that typically agglomerates into intracellular bodies (inclusion bodies) within the host cell. The fusion protein comprises at least one portion comprising an inclusion body tag and at least one portion comprising the polypeptide of interest. In one aspect, the protein/polypeptides of interest are separated from the inclusion body tags using cleavable peptide linker elements. Using the present method, inclusion body tags of about 10 to about 50 amino acids in length are identified from portions of a large insoluble protein. The length of the inclusion body tags identified using the present method are about 10 to about 50 amino acids in length, preferably 10 to about 35 amino acids in length, more preferably 10 to about 25 amino acids in length, and more preferably 12 to 15 amino acids in length.

As used herein, “cleavable linker elements”, “peptide linkers”, and “cleavable peptide linkers” will be used interchangeably and refer to cleavable peptide segments typically found between inclusion body tags and the peptide of interest. After the inclusion bodies are separated and/or partially-purified or purified from the cell lysate, the cleavable linker elements can be cleaved chemically and/or enzymatically to separate the inclusion body tag from the peptide of interest. The peptide of interest can then be isolated from the inclusion body tag, if necessary. In one embodiment, the inclusion body tag(s) and the peptide of interest exhibit different solubilities in a defined medium (typically an aqueous medium), facilitating separation of the inclusion body tag from the protein/polypeptide of interest. In a preferred embodiment, the inclusion body tag is insoluble in an aqueous solution while the protein/polypeptide of interest is appreciably soluble in an aqueous solution. The pH, temperature, and/or ionic strength of the aqueous solution can be adjusted to facilitate recovery of the peptide of interest. In a preferred embodiment, the differential solubility between the inclusion body tag and the peptide of interest occurs in an aqueous solution having a pH of 5 to 10 and a temperature range of 15 to 50° C. The cleavable peptide linker may be from 1 to about 50 amino acids, preferably from 1 to about 20 amino acids in length. An example of a cleavable peptide linker is provided by SEQ ID NO: 446 (Caspase-3 cleavage sequence). The cleavable peptide linkers may be incorporated into the fusion proteins using any number of techniques well known in the art.

As used herein, the term “dispersant” as used herein refers to a substance that stabilizes the formation of a colloidal solution of solid pigment particles in a liquid medium. As used herein, the term “triblock dispersant” to a pigment dispersant that consists of three different units or blocks, each serving a specific function. In the present examples, a synthetic peptide encoding a peptide-based triblock dispersant was used as the “peptide of interest” to evaluate the performance of the present inclusion body tags (U.S. Ser. No. 10/935,254).

As used herein, the term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). In a further embodiment, the definition of “operably linked” may also be extended to describe the products of chimeric genes, such as fusion proteins. As such, “operably linked” will also refer to the linking of an inclusion body tag to a peptide of interest to be produced and recovered. The inclusion body tag is “operably linked” to the peptide of interest if upon expression the fusion protein is insoluble and accumulates it inclusion bodies in the expressing host cell. In a preferred embodiment, the fusion peptide will include at least one cleavable peptide linker useful in separating the inclusion body tag from the peptide of interest. In a further preferred embodiment, the cleavable linker is an acid cleavable aspartic acid—proline dipeptide (D-P) moiety (see INK101DP; SEQ ID NO: 18). The cleavable peptide linkers may be incorporated into the fusion proteins using any number of techniques well known in the art.

As used herein, the term “in a combinatorial fashion” or “combinatorially” means an action, method or process wherein combinations of different, but structurally related molecules are assembled from combinations and/or arrangements of elements in sets. As shown in the present examples, a library of genetic constructs encoding fusion proteins were prepared by combining various portions of a large, insoluble protein (“the peptide tag”) with a short peptide of interest. Each of the constructs was expressed in an appropriate host cell and assessed for inclusion body formation.

As used herein, the term “tag scanning” or “sequence scanning” will be used to refer to the present method of assaying a library of short, overlapping peptide tags derived from a large, insoluble protein for their ability to promote insoluble fusion peptide formation when operably linked a short peptide of interest. In the present method, a library of genetic constructs (chimeric genes) are prepared encoding fusion peptides comprising at least one first portion and at least one second portion wherein said first portion comprises a 10 to 50 contiguous amino acid sequence derived from a large, insoluble protein fused to said second portion comprising a short peptide of interest. In a preferred aspect, the first portion comprises 10 to 35 contiguous amino acids, preferably 10 to 25 contiguous amino acid, and more preferably 12-15 contiguous amino acids from a portion of a large insoluble protein.

As used herein, “contiguous amino acids” means a peptide of a defined length comprising an amino acid sequence identical to a portion of a large, insoluble protein from which the sequence was derived.

As used herein, the terms “large insoluble protein”, “insoluble full-length protein”, and “insoluble carrier protein” will be used interchangeably and used to describe (1) a protein reported in the art to be insoluble under normal physiological conditions (i.e., when expressed in a suitable host cell) or (2) a protein having high homology to a protein reported to typically be insoluble under normal physiological conditions. Recombinant peptide production using a large, insoluble carrier protein is known in the art. However, the production efficiency for short peptides of interest is adversely affected when fused to a large, insoluble carrier protein (i.e., the short peptide of interest comprises only a small weight percent of the total fusion protein). As such, the present method is used to identify small portions of the larger insoluble protein that have the ability to induce inclusion body formation. In one aspect, the large insoluble protein of interest is at least 100 amino acids in length, preferably at least 125 amino acids in length, more preferably at least 150 amino acids in length, and most preferably at least 175 amino acids in length. As exemplified herein, two different large, insoluble proteins (cystatin and zein) were evaluated using the present method and found to contain regions suitable for use in preparing inclusion body tags.

As used herein, the terms “fusion protein”, “fusion peptide”, “chimeric protein”, and “chimeric peptide” will be used interchangeably and will refer to a polymer of amino acids (peptide, oligopeptide, polypeptide, or protein) comprising at least one first portion and at least one second portion, each portion comprising a distinct function. The first portion of the fusion peptide comprises at least one of the present inclusion body tags. The second portion comprises at least one peptide of interest. In a preferred embodiment, the fusion protein additionally includes at least one additional portion comprising at least one cleavable peptide linker that facilitates cleavage (chemical and/or enzymatic) and separation of the inclusion body tag(s) and the peptide(s) of interest.

Means to prepare peptides (inclusion body tags, cleavable peptide linkers, peptides of interest, and fusion peptides) are well known in the art (see, for example, Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). The various components of the fusion peptides (inclusion body tag, peptide of interest, and the cleavable linker) described herein can be combined using carbodiimide coupling agents (see for example, Hermanson, Greg T., Bioconjugate Techniques, Academic Press, New York (1996)), diacid chlorides, diisocyanates and other difunctional coupling reagents that are reactive to terminal amine and/or carboxylic acid groups on the peptides. However, chemical synthesis is often limited to peptides of less than about 50 amino acids length due to cost and/or impurities. In a preferred embodiment, the entire peptide reagent is prepared using recombinant DNA and molecular cloning techniques.

As used herein, the terms “polypeptide” and “peptide” will be used interchangeably to refer to a polymer of two or more amino acids joined together by a peptide bond, wherein the peptide is of unspecified length, thus, peptides, oligopeptides, polypeptides, and proteins are included within the present definition. In one aspect, this term also includes post expression modifications of the polypeptide, for example, glycosylations, acetylations, phosphorylations and the like. Included within the definition are, for example, peptides containing one or more analogues of an amino acid or labeled amino acids and peptidomimetics.

As used herein, the terms “polypeptide of interest”, “peptide of interest”, “short peptide of interest”, “targeted protein”, “targeted polypeptide”, and “targeted peptide” will be used interchangeably and refer to a peptide having a defined activity or use that may be expressed by the genetic machinery of a host cell. In one aspect, the present method is useful for identifying inclusion body tags suitable for expressing short, soluble, peptides of interest in an insoluble form (i.e. an insoluble fusion peptide). In another aspect, the short peptide of interest is less than 100 amino acids in length, preferably less than 75 amino acids in length, more preferably less than 50 amino acids in length, and more preferably less than 35 amino acids in length.

As used herein, the terms “bioactive” and “peptide of interest activity” are used interchangeably and refer to the activity or characteristic associated with the peptide of interest. The bioactive peptides may be used in a variety of applications including, but not limited to curative agents for diseases (e.g., insulin, interferon, interleukins, anti-angiogenic peptides (U.S. Pat. No. 6,815,426), and polypeptides that bind to defined cellular targets such as receptors, channels, lipids, cytosolic proteins, and membrane proteins, to name a few), peptides having antimicrobial activity, peptides having an affinity for a particular material (e.g., hair binding polypeptides, skin binding polypeptides, nail binding polypeptides, cellulose binding polypeptides, polymer binding polypeptides, clay binding polypeptides, silicon binding polypeptides, carbon nanotube binding polypeptides, and peptides that have an affinity for particular animal or plant tissues) for targeted delivery of benefit agents.

As used herein, the “benefit agent” refers to a molecule that imparts a desired functionality to the complex for a defined application. The benefit agent may be peptide of interest itself or may be one or more molecules bound to (covalently or non-covalently), or associated with, the peptide of interest wherein the binding affinity of the targeted polypeptide is used to selectively target the benefit agent to the targeted material. In another embodiment, the targeted polypeptide comprises at least one region having an affinity for at least one target material (e.g., biological molecules, polymers, hair, skin, nail, other peptides, etc.) and at least one region having an affinity for the benefit agent (e.g., pharmaceutical agents, pigments, conditioners, dyes, fragrances, etc.). In another embodiment, the peptide of interest comprises a plurality of regions having an affinity for the target material and a plurality of regions having an affinity for the benefit agent. In yet another embodiment, the peptide of interest comprises at least one region having an affinity for a targeted material and a plurality of regions having an affinity for a variety of benefit agents wherein the benefit agents may be the same of different. Examples of benefits agents may include, but are not limited to conditioners for personal care products, pigments, dye, fragrances, pharmaceutical agents (e.g., targeted delivery of cancer treatment agents), diagnostic/labeling agents, ultraviolet light blocking agents (i.e., active agents in sunscreen protectants), and antimicrobial agents (e.g., antimicrobial peptides), to name a few.

As used herein, an “inclusion body” is an intracellular amorphous deposit comprising aggregated protein found in the cytoplasm of a cell. Peptides of interest that are typically soluble with the host cell and/or cell lysates can be fused to one or more of the short inclusion body tags to facilitate formation of an insoluble fusion protein. In an alternative embodiment, the peptide of interest may be partially insoluble in the host cell, but produced at relatively lows levels where significant inclusion body formation does not occur. In a further embodiment, fusion of the peptide of interest to one or more inclusion body tags (IBTs) increases the amount of protein produced in the host cell. Formation of the inclusion body facilitates simple and efficient isolation of the fusion peptide from the cell lysate using techniques well known in the art such as centrifugation and filtration. The isolated fusion peptide may be further processed using any number of common purification techniques well known in the art (precipitation, extraction, ion exchange, chromatographic techniques, etc.) to isolate the desired peptide of interest. The fusion protein typically includes one or more cleavable peptide linkers used to separate the protein/polypeptide of interest from the inclusion body tag(s). The cleavable peptide linker is designed so that the inclusion body tag(s) and the protein/polypeptide(s) of interest can be easily separated by cleaving the linker element. The peptide linker can be cleaved chemically (e.g., acid hydrolysis) or enzymatically (i.e., use of a protease/peptidase that preferentially recognizes an amino acid cleavage site and/or sequence within the cleavable peptide linker).

As used herein, the term “solubility” refers to the amount of a substance that can be dissolved in a unit volume of a liquid under specified conditions. In the present application, the term “solubility” is used to describe the ability of a peptide (inclusion body tag, peptide of interest, or fusion peptides) to be resuspended in a volume of solvent, such as a biological buffer. In one aspect, the substance (peptide, fusion peptide, inclusion body tags, etc.) is “insoluble” when less than 5 mg can be dissolved in 100 mL of solvent (e.g. an aqueous matrix such as biological buffer). In one embodiment, the peptides targeted for production (“peptides of interest”) are normally soluble in the cell and/or cell lysate under normal physiological conditions. Fusion of one or more inclusion body tags (IBTs) to the target peptide results in the formation of a fusion peptide that is insoluble under normal physiological conditions, resulting in the formation of inclusion bodies. In one embodiment, the peptide of interest is insoluble in an aqueous matrix having a pH range of 5-12, preferably 6-10; and a temperature range of 5° C. to 50° C., preferably 10° C. to 40° C. Fusion of the peptide of interest to at least one of the present inclusion body tags results in the formation of an insoluble fusion protein that agglomerates into at least one inclusion body under normal physiological conditions.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Miscellaneous | Xaa | X |
| (or as defined herein) | | |

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences (including coding regions engineered to encode fusion peptides) that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

As used herein, the term “genetic construct” refers to a series of contiguous nucleic acids useful for modulating the genotype or phenotype of an organism. Non-limiting examples of a genetic constructs include, but are not limited to a nucleic acid molecule, an open reading frame, a gene, a coding region, a plasmid, and the like. Typically, the genetic construct will include a chimeric gene encoding a fusion peptide, said chimeric gene comprising a coding region operably linked to suitable 5′ and 3′ regulatory regions. Given the structures of (1) the inclusion body tag and (2) the peptide of interest, it is well within the skill of one in the art to assemble an expressible genetic construct encoding the desired fusion peptide.

As used herein, the term “expression ranking” means the relative yield of insoluble fusion protein estimated visually and scored on a relative scale of 0 (no insoluble fusion peptide) to 3 (highest yield of insoluble fusion peptide). As described in the present examples, the relative yield of insoluble fusion protein was estimated visually from stained polyacrylamide gels.

As used herein, the term “transformation” refers to the transfer of a nucleic acid fragment into the genome of a host organism, resulting in genetically stable inheritance. As used herein, the host cell's genome is comprised of chromosomal and extrachromosomal (e.g., plasmid) genes. Host organisms containing the transformed nucleic acid fragments are referred to as “transgenic” or “recombinant” or “transformed” organisms.

As used herein, the term “host cell” refers to cell which has been transformed or transfected, or is capable of transformation or transfection by an exogenous polynucleotide sequence.

As used herein, the terms “plasmid”, “vector” and “cassette” refer to an extrachromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

Standard recombinant DNA and molecular cloning techniques used herein are well known in the art and are described by Sambrook, J., Fritsch, E. F. and Maniatis, T., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

### Insoluble Protein Sequence Scanning

Many large carrier proteins have been used to produce insoluble fusion proteins. Examples of these proteins include β-galactosidase, glutathione-S-transferase, bacteriophage T4 gp55 protein, and bacterial ketosteroid isomerase, to name a few. However, the use of a large carrier protein for recombinant peptide production significantly reduces the overall production efficiency, especially when the peptide of interest is small (<100 amino acids). As such, the peptide of interest is only a small percentage of the total mass of the purified fusion protein. There is a need to identify short peptide tags capable of inducing insoluble fusion peptide formation.

The present method provides short peptide tags (“inclusion body tags”) suitable for preparing insoluble fusion peptides. The present method identifies portions and/or regions of larger, insoluble proteins that are suitable for use an inclusion body tags.

In general, a library of genetic constructs is prepared encoding a library of fusion peptides. Each fusion peptide comprises at least two portions. The first portion comprises a 10-50 contiguous amino acid sequence from a larger, insoluble protein. The second portion comprises a short peptide of interest that is typically soluble and/or difficult to produce due to the host cell's endogenous proteolytic activity. The library is constructed such that short, 10-50 contiguous amino acid peptide tags are generated beginning at the N-terminal region of the full-length insoluble protein, extending to the C-terminal end of the insoluble full-length protein, each peptide tag overlapping the next peptide tag in the library by about 3 to about 10 amino acids.

The genetic constructs encoding the various members of the fusion peptide library are transformed and expressed in an appropriate host cell. Host cells comprising the fusion peptides are evaluated for inclusion body formation. The sequences of the peptide tags capable of inducing inclusion body formation are compared to the sequence of the insoluble full-length protein.

Preferably, each of the amino acid residues within the larger, insoluble peptide will be found within at least one of the members of the peptide tag library. In another preferred aspect, each amino acid from the larger, insoluble full-length protein will be represented in a plurality of overlapping members within the tag library. In this way, the entire sequence of the insoluble full-length protein is evaluated for suitable short inclusion body tags. Regions of the insoluble full-length protein that produce short peptide tags having inclusion body forming ability can be identified and refined by comparing the effective inclusion body forming tags against the sequence of the insoluble full-length protein. As shown in the present examples, suitable regions will typically be represented by multiple tags within the library (i.e., the inclusion body tag sequences will typically overlap to some extent).

The insoluble full-length protein refers to any protein reported to be insoluble under normal physiological conditions or a protein believed to insoluble based on homology to another insoluble protein. In another aspect, the selected protein used to prepare the library of short peptide tags has significant homology to a natural full-length protein reported in the art to be insoluble. In one embodiment, “significant homology” means a protein having at least 60%, preferably at least 70%, more preferably at least 80%, even more preferably at least 90%, and most preferably at least 95% amino acid sequence identify to a previously reported full-length insoluble protein. In a yet another aspect, the full-length protein is an insoluble protein found in nature or a derivative of the full-length protein found in nature sharing high homology over at least 100 amino acids to the natural protein. In another embodiment, proteins having “significant homology” to an insoluble full-length protein may also be identified by structural similarities between their respective gene sequences (i.e. coding regions). A common tool to identify nucleic acid molecules sharing significant homology is hybridization (Maniatis, supra). The skilled artisan recognizes that substantially similar nucleic acid sequences encoding full-length insoluble proteins can be defined by their ability to hybridize, under highly stringent conditions (0.1×SSC, 0.1% SDS, 65° C. and washed with 2×SSC, 0.1% SDS followed by 0.1×SSC, 0.1% SDS, 65° C.), with the target sequence.

In one aspect, the library of peptide tags is prepared from a full-length insoluble protein that is typically at least 100 amino acids in length, preferably at least 125 amino acids in length, more preferably at least 150 amino acids in length, and most preferably at least 175 amino acids in length.

In one aspect, the library of peptide tags is prepared to ensure that the overlapping members of the library cover at least 90% of the entire length of the full-length insoluble protein. In a highly preferred aspect, the library of peptide tags covers the entire length of the full-length insoluble protein.

The peptide tags are designed to represent a 10 to 50 contiguous amino acid portion of the full-length insoluble protein. In one aspect, the members of the peptide tag library are 10 to about 35 amino acids in length, more preferably 10 to about 25 amino acids in length, and more preferably 12 to 15 amino acids in length.

The library of peptide tags library is designed so that at least each peptide tag overlaps with another peptide tags by about 3 to about 10 amino acids, preferably overlapping from 3 to 10 amino acids, more preferably overlapping by about 3 to 6 about amino acids, and most preferably overlapping by about 5 amino acids. The use of overlapping tags enables one to refine and identify those regions suitable for preparing short inclusion body tags.

The structure of short peptide tags capable of inducing inclusion body formation is somewhat unpredictable. As such, the present method simplifies a process to identify the regions within larger, insoluble proteins responsible for inducing inclusion body formation. In one aspect, the structural information obtained using the present methodology can be used to develop a database inclusion body tags. In a further aspect, the information within the database is used to design further inclusion body tags.

### Inclusion Body Tags

Exemplified herein are inclusion body tags prepared and identified by the present method. The peptide tags were derived from the Daucus carota cystatin protein (GenBank® accession No. BAA20464; SEQ ID NO: 160) or the Zea mays zein protein (GenBank® AAP32017; SEQ ID NO: 20). Each of these proteins was selected as the starting material for preparation of a library of putative inclusion body tags. Several overlapping series of 12 to 15 amino acid long peptides were prepared and evaluated from each protein as potential inclusion body tags. The library was prepared by synthesizing and fusing short peptides (12-15 contiguous amino acids) identical to various sections of each respective protein to a soluble peptide of interest. Expression analysis identified a two regions of the cystatin protein (amino acid residues 1-28 or 45-133 of SEQ ID NO: 160) and a central region of the zein protein (amino acid residues 76-175 of SEQ ID NO: 20) that were particularly suitable for the preparation of short inclusion body tags. Short inclusion body tags prepared from the region(s) of the respective proteins were able to induce inclusion body formation (i.e. form insoluble fusion peptides) when fused to a short peptide of interest.

Each of the fusion tags prepared by the present method was fused to a standard peptide of interest (a modified version of the TBP101 peptide (INK101DP) incorporating an acid cleavable aspartic acid—proline moiety useful in separating the peptide of interest from the inclusion body tag; see Example 1). TBP101 (when not linked to an inclusion body tag) is a short, soluble, peptide of interest in the present test system. Each genetic construct was recombinantly expressed in an appropriate host cell and evaluated for insoluble fusion peptide formation.

Using the present method, a family zein-derived inclusion body tags were identified having an amino acid sequence selected from the group consisting of SEQ ID NOs: 116, 117, 119, 121, 125, 131, 132, 133, 135, 145, 147, 148, 149, 150, 154, 155, 157, and 158.

The present method was repeated using the Daucus carota cystatin protein (SEQ ID NO: 160) resulting in the identification of a family of cystatin-derived inclusion body tags having an amino acid sequence selected from the group consisting of SEQ ID NOs: 223, 224, 227, 228, 229, 230, 231, 232, 233, 238, 240, 242, 247, 248, 249, 252, and 253.

In another aspect, the present method may be used to scan a library of genetic constructs that are also designed to include at least one cleavable peptide linker useful in separating the peptide of interest from the fusion peptide. The cleavable peptide linker can be an enzymatic cleavage sequence and/or a chemical cleavage sequence. In another preferred embodiment, the cleavable peptide linker comprises at least one acid cleavable aspartic acid—proline moiety (for example, see the INK101DP peptide; SEQ ID NO: 18).

### Expressible Peptides of Interest

The peptide of interest (“expressible peptide”) is one that is appreciably soluble in the host cell and/or host cell liquid lysate under normal physiological conditions. In a preferred aspect, the peptides of interest are generally short (<100 amino acids in length) and difficult to produce in sufficient amounts due to proteolytic degradation. Fusion of the peptide of interest to at least one inclusion body forming tag identified by the present method creates a fusion peptide that is insoluble in the host cell and/or host cell lysate under normal physiological conditions. Production of the peptide of interest is typically increased when expressed and accumulated in the form of an insoluble inclusion body. Production of the peptide of interest in an insoluble form facilitates simple isolation from the cell lysate using procedures such as centrifugation or filtration.

The length of the peptide of interest may vary as long as (1) the peptide is appreciably soluble in the host cell and/or cell lysate, and/or (2) the amount of the targeted peptide produced is significantly increased when expressed in the form of an insoluble fusion peptide/inclusion body (i.e. expression in the form of a fusion protein protect the peptide of interest from proteolytic degradation). Typically the peptide of interest is less than 200 amino acids in length, preferably less than 100 amino acids in length, more preferably less than 75 amino acids in length, even more preferably less than 50 amino acids in length, and most preferably less than 25 amino acids in length.

The function of the peptide of interest is not limited by the present method and may include, but is not limited to bioactive molecules such as curative agents for diseases (e.g., insulin, interferon, interleukins, peptide hormones, anti-angiogenic peptides, and peptides that bind to and affect defined cellular targets such as receptors, channels, lipids, cytosolic proteins, and membrane proteins; see U.S. Pat. No. 6,696,089,), peptides having an affinity for a particular material (e.g., biological tissues, biological molecules, hair binding peptides (U.S. patent application Ser. No. 11/074,473; WO 0179479; U.S. Patent Application Publication No. 2002/0098524; U.S. Patent Application Publication No. 2003/0152976; WO 04048399; U.S. Provisional Patent Application No. 60/721,329; and U.S. Provisional Patent Application No. 60/790,149)., skin binding peptides (U.S. patent application Ser. No. 11/069,858; WO 2004/000257; and U.S. Provisional Patent Application No. 60/790,149), nail binding peptides (U.S. Provisional Patent Application No. 60/790,149), cellulose binding peptides, polymer binding peptides (U.S. Provision Patent Application Nos. 60/750,598, 60/750,599, 60/750,726, 60/750,748, and 60/750,850), clay binding peptides, silicon binding peptides, and carbon nanotube binding peptides) for targeted delivery of at least one benefit agent (see U.S. patent application Ser. No. 10/935,642; U.S. patent application Ser. No. 11/074,473; and U.S. Provisional Patent Application No. 60/790,149).

In a preferred aspect, the peptide of interest is selected from the group of hair binding peptides (U.S. patent application Ser. No. 11/074,473; WO 0179479; U.S. Patent Application Publication No. 2002/0098524; Janssen et al., U.S. Patent Application Publication No. 2003/0152976; WO 04048399; U.S. Provisional Patent Application No. 60/721,329; and U.S. Provisional Patent Application No. 60/790,149), skin binding peptides (U.S. patent application Ser. No. 11/069,858; WO 2004/000257; and U.S. Provisional Patent Application No. 60/790,149), nail binding peptides (U.S. Provisional Patent Application No. 60/790,149), antimicrobial peptides (U.S. Provisional Patent Application No. 60/790,149), and polymer binding peptides (U.S. Provision Patent Application Nos. 60/750,598, 60/750,599, 60/750,726, 60/750,748, and 60/750,850). In another preferred aspect, the hair binding peptide is selected from the group consisting of SEQ ID NOs: 262-354; the skin binding peptide is selected from the group consisting of SEQ ID NOs: 254-261; the nail binding peptide is selected from the group consisting of SEQ ID NOs: 355-356; the antimicrobial peptide is selected from the group consisting of SEQ ID NOs: 357-385; the pigment binding peptide selected from the group consisting of SEQ ID NOs: 386-411; and the polymer binding peptide is selected from the group consisting of SEQ ID NOs: 412-445.

As used herein, the “benefit agent” refers to a molecule that imparts a desired functionality to a target material (e.g., hair, skin, etc.) for a defined application (U.S. patent application Ser. No. 10/935,642; U.S. patent application Ser. No. 11/074,473; and U.S. Patent Application 60/790,149 for a list of typical benefit agents such as conditioners, pigments/colorants, fragrances, etc.). The benefit agent may be peptide of interest itself or may be one or more molecules bound to (covalently or non-covalently), or associated with, the peptide of interest wherein the binding affinity of the peptide of interest is used to selectively target the benefit agent to the targeted material. In another embodiment, the peptide of interest comprises at least one region having an affinity for at least one target material (e.g., biological molecules, polymers, hair, skin, nail, other peptides, etc.) and at least one region having an affinity for the benefit agent (e.g., pharmaceutical agents, antimicrobial agents, pigments, conditioners, dyes, fragrances, etc.). In another embodiment, the peptide of interest comprises a plurality of regions having an affinity for the target material and a plurality of regions having an affinity for one or more benefit agents. In yet another embodiment, the peptide of interest comprises at least one region having an affinity for a targeted material and a plurality of regions having an affinity for a variety of benefit agents wherein the benefit agents may be the same of different. Examples of benefits agents may include, but are not limited to conditioners for personal care products, pigments, dye, fragrances, pharmaceutical agents (e.g., targeted delivery of cancer treatment agents), diagnostic/labeling agents, ultraviolet light blocking agents (i.e., active agents in sunscreen protectants), and antimicrobial agents (e.g., antimicrobial peptides), to name a few.

### Cleavable Peptide Linkers

The present method provides short inclusion body tags useful in preparing insoluble fusion peptides. Given an inclusion body tag identified by the present method, it is well within the skill of one in the art to prepare genetic constructs encoding fusion peptides/proteins comprising the peptide of interest. In a preferred embodiment, the fusion peptide will include at least one cleavable peptide linker separating the inclusion body tag(s) from the peptide(s) of interest.

The use of cleavable peptide linkers is well known in the art. The cleavable sequence facilitates separation of the inclusion body tag(s) from the peptide(s) of interest. In one embodiment, the cleavable sequence may be provided by a portion of the inclusion body tag and/or the peptide of interest (e.g., inclusion of an acid cleavable aspartic acid—proline moiety). In a preferred embodiment, the cleavable sequence is provided by including (in the fusion peptide) at least one cleavable peptide linker between the inclusion body tag and the peptide of interest.

Means to cleave the peptide linkers are well known in the art and may include chemical hydrolysis, enzymatic cleavage agents, and combinations thereof. In one embodiment, one or more chemically cleavable peptide linkers are included in the fusion construct to facilitate recovery of the peptide of interest from the inclusion body fusion protein. Examples of chemical cleavage reagents include cyanogen bromide (cleaves methionine residues), N-chloro succinimide, iodobenzoic acid or BNPS-skatole [2-(2-nitrophenylsulfenyl)-3-methylindole] (cleaves tryptophan residues), dilute acids (cleaves at aspartyl-prolyl bonds), and hydroxylamine (cleaves at asparagine-glycine bonds at pH 9.0); see Gavit, P. and Better, M., J. Biotechnol., 79:127-136 (2000); Szoka et al., DNA, 5(1):11-20 (1986); and Walker, J. M., The Proteomics Protocols Handbook, 2005, Humana Press, Totowa, N.J.)). In a preferred embodiment, one or more aspartic acid—proline acid cleavable recognition sites (i.e., a cleavable peptide linker comprising one or more D-P dipeptide moieties) are included in the fusion protein construct to facilitate separation of the inclusion body tag(s) form the peptide of interest. In another embodiment, the fusion peptide may include multiple regions encoding peptides of interest separated by one or more cleavable peptide linkers.

In another embodiment, one or more enzymatic cleavage sequences are included in the fusion protein construct to facilitate recovery of the peptide of interest. Proteolytic enzymes and their respective cleavage site specificities are well known in the art. In a preferred embodiment, the proteolytic enzyme is selected to specifically cleave only the peptide linker separating the inclusion body tag and the peptide of interest. Examples of enzymes useful for cleaving the peptide linker include, but are not limited to Arg-C proteinase, Asp-N endopeptidase, chymotrypsin, clostripain, enterokinase, Factor Xa, glutamyl endopeptidase, Granzyme B, Achromobacter proteinase I, pepsin, proline endopeptidase, proteinase K, Staphylococcal peptidase I, thermolysin, thrombin, trypsin, and members of the Caspase family of proteolytic enzymes (e.g. Caspases 1-10) (Walker, J. M., supra). An example of a cleavage site sequence is provided by SEQ ID NO: 446 (Caspase-3 cleavage site; Thornberry et al., J. Biol. Chem., 272:17907-17911 (1997) and Tyas et al., EMBO Reports, 1(3):266-270 (2000)).

Typically, the cleavage step occurs after the insoluble inclusion bodies and/or insoluble fusion peptides are isolated from the cell lysate. The cells can be lysed using any number of means well known in the art (e.g. mechanical, enzymatic, and/or chemical lysis). Methods to isolate the insoluble inclusion bodies/fusion peptides from the cell lysate are well known in the art (e.g., centrifugation, filtration, and combinations thereof). Once recovered from the cell lysate, the insoluble inclusion bodies and/or fusion peptides can be treated with a cleavage agent (chemical or enzymatic) to cleavage the inclusion body tag from the peptide of interest. In one embodiment, the fusion protein and/or inclusion body is diluted and/or dissolved in a suitable solvent prior to treatment with the cleavage agent. In a further embodiment, the cleavage step may be omitted if the inclusion body tag does not interfere with the activity of the peptide of interest.

After the cleavage step, and in a preferred embodiment, the peptide of interest can be separated and/or isolated from the fusion protein and the inclusion body tags based on a differential solubility of the components. Parameters such as pH, salt concentration, and temperature may be adjusted to facilitate separation of the inclusion body tag from the peptide of interest. In one embodiment, the peptide of interest is soluble while the inclusion body tag and/or fusion protein is insoluble in the defined process matrix (typically an aqueous matrix). In another embodiment, the peptide of interest is insoluble while the inclusion body tag is soluble in the defined process matrix.

In an alternate embodiment, the peptide of interest may be further purified using any number of well known purification techniques in the art such as ion exchange, gel purification techniques, and column chromatography (see U.S. Pat. No. 5,648,244), to name a few.

### Fusion Peptides

The present method identifies short peptide tags useful for recombinant production of insoluble chimeric polypeptides (“fusion peptides” or “fusion proteins”). Synthesis and expression of genetic constructs encoding fusion peptides is well known to one of skill.

The fusion peptides will include at least one of the inclusion body tags identified by the present method (IBTs) operably linked to at least one peptide of interest. Typically, the fusion peptides will also include at least one cleavable peptide linker having a cleavage site between the inclusion body tag and the peptide of interest. In one embodiment, the inclusion body tag may include a cleavage site whereby inclusion of a separate cleavable peptide linker may not be necessary. In a preferred embodiment, the cleavage method is chosen to ensure that the peptide of interest is not adversely affected by the cleavage agent(s) employed. In a further embodiment, the peptide of interest may be modified to eliminate possible cleavage sites with the peptide so long as the desired activity of the peptide is not adversely altered.

One of skill in the art will recognize that the elements of the fusion protein can be structured in a variety of ways. Typically, the fusion protein will include at least one IBT, at least one peptide of interest (P01), and at least one cleavable linker (CL) located between the IBT and the POI. The inclusion body tag may be organized as a leader sequence or a terminator sequence relative to the position of the peptide of interest within the fusion peptide. In another embodiment, a plurality of IBTs, POIs, and CLs are used when engineering the fusion peptide. In a further embodiment, the fusion peptide may include a plurality of IBTs (as defined herein), POIs, and CLs that are the same or different.

The fusion peptide should be insoluble in an aqueous matrix at a temperature of 10° C. to 50° C., preferably 10° C. to 40° C. The aqueous matrix typically comprises a pH range of 5 to 12, preferably 6 to 10, and most preferably 6 to 8. The temperature, pH, and/or ionic strength of the aqueous matrix can be adjusted to obtain the desired solubility characteristics of the fusion peptide/inclusion body.

### Method to Make a Peptide of Interest Using Insoluble Fusion Peptides

The inclusion body tags provided by the present method are used to make fusion peptides that form inclusion bodies within the production host. This method is particularly attractive for producing significant amounts of soluble peptide of interest that (1) are difficult to isolation from other soluble components of the cell lysate and/or (2) are difficult to product in significant amounts within the target production host.

Typically, the peptide of interest is fused to at least one of the present inclusion body tags. Expression of the genetic construct encoding the fusion protein produces an insoluble form of the peptide of interest that accumulates in the form of inclusion bodies within the host cell. The host cell is grown for a period of time sufficient for the insoluble fusion peptide to accumulate within the cell.

The host cell is subsequently lysed using any number of techniques well known in the art. The insoluble fusion peptide/inclusion bodies are then separated from the soluble components of the cell lysate using a simple and economical technique such as centrifugation, filtration, and combinations thereof. The insoluble fusion peptide/inclusion body can then be further processed in order to isolate the peptide of interest. Typically, this will include resuspension of the fusion peptide/inclusion body in a liquid matrix suitable for cleaving the fusion peptide followed by separation of the inclusion body tag from the peptide of interest. The fusion protein is typically designed to include a cleavable peptide linker separating the inclusion body tag from the peptide of interest. The cleavage step can be conducted using any number of techniques well known in the art (chemical cleavage, enzymatic cleavage, and combinations thereof). The peptide of interest is subsequently separated from the inclusion body tag(s) and/or fusion peptides using any number of techniques well known in the art (centrifugation, filtration, precipitation, column chromatography, etc.). Preferably, the peptide of interest (once cleaved from fusion peptide) has a solubility that is significantly different than that of the inclusion body tag and/or remaining fusion peptide.

### Transformation and Expression

Given the structures of the various components (i.e., an inclusion body tag, a peptide of interest, a cleavable peptide linker, etc.), it is well within the skill of one in the art to prepare expressible genetic constructs suitable for transformation and expression in a chosen host cell. The expressible genetic construct can be chromosomally (i.e., chromosomally integrated) and/or extrachromosomally expressed (e.g., an expression plasmid). Typically, an expression vector comprises sequences directing transcription and translation of the relevant chimeric gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host.

Initiation control regions or promoters, which are useful to drive expression of the genetic constructs encoding the fusion peptides in the desired host cell, are numerous and familiar to those skilled in the art. Virtually any promoter capable of driving these constructs is suitable for the present invention including but not limited to CYC1, HIS3, GAL1, GAL 10, ADH1, PGK, PHO5, GAPDH, ADC1, TRP1, URA3, LEU2, ENO, TPI (useful for expression in Saccharomyces); AOX1 (useful for expression in Pichia); and lac, ara (pBAD), tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

Preferred host cells for expression of the fusion peptides are microbial hosts that can be found broadly within the fungal or bacterial families and which grow over a wide range of temperature, pH values, and solvent tolerances. For example, it is contemplated that any of bacteria, yeast, and filamentous fungi will be suitable hosts for expression of the present nucleic acid molecules encoding the fusion peptides. Because of transcription, translation, and the protein biosynthetic apparatus is the same irrespective of the cellular feedstock, genes are expressed irrespective of the carbon feedstock used to generate the cellular biomass. Large-scale microbial growth and functional gene expression may utilize a wide range of simple or complex carbohydrates, organic acids and alcohols (i.e. methanol), saturated hydrocarbons such as methane or carbon dioxide in the case of photosynthetic or chemoautotrophic hosts. However, the functional genes may be regulated, repressed or depressed by specific growth conditions, which may include the form and amount of nitrogen, phosphorous, sulfur, oxygen, carbon or any trace micronutrient including small inorganic ions. In addition, the regulation of functional genes may be achieved by the presence or absence of specific regulatory molecules that are added to the culture and are not typically considered nutrient or energy sources. Growth rate may also be an important regulatory factor in gene expression. Examples of host strains include, but are not limited to fungal or yeast species such as Aspergillus, Trichoderma, Saccharomyces, Pichia, Candida, Hansenula, or bacterial species such as Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. Preferred bacterial host strains include Escherichia and Bacillus. In a highly preferred aspect, the host strain is Escherichia coli.

### Fermentation Media

Fermentation media in the present invention must contain suitable carbon substrates. Suitable substrates may include but are not limited to monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally the carbon substrate may also be one-carbon substrates such as carbon dioxide, or methanol for which metabolic conversion into key biochemical intermediates has been demonstrated. In addition to one and two carbon substrates methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1 Compd., [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, preferred carbon substrates are glucose, fructose, and sucrose.

In addition to an appropriate carbon source, fermentation media must contain suitable minerals, salts, cofactors, buffers and other components, known to those skilled in the art, suitable for the growth of the cultures and promotion of the expression of the present fusion peptides.

### Culture Conditions

Suitable culture conditions can be selected dependent upon the chosen production host. Typically, cells are grown at a temperature in the range of about 25° C. to about 40° C. in an appropriate medium. Suitable growth media in the present invention are common commercially prepared media such as Luria Bertani (LB) broth, Sabouraud Dextrose (SD) broth or Yeast medium (YM) broth. Other defined or synthetic growth media may also be used and the appropriate medium for growth of the particular microorganism will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation are typically between pH 5.0 to pH 9.0, where pH 6.0 to pH 8.0 is preferred.

Fermentations may be performed under aerobic or anaerobic conditions wherein aerobic conditions are preferred.

### Industrial Batch and Continuous Fermentations

A classical batch fermentation is a closed system where the composition of the medium is set at the beginning of the fermentation and not subject to artificial alterations during the fermentation. Thus, at the beginning of the fermentation the medium is inoculated with the desired organism or organisms, and fermentation is permitted to occur without adding anything to the system. Typically, a “batch” fermentation is batch with respect to the addition of carbon source and attempts are often made at controlling factors such as pH and oxygen concentration. In batch systems the metabolite and biomass compositions of the system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of end product or intermediate.

A variation on the standard batch system is the Fed-Batch system. Fed-Batch fermentation processes are also suitable in the present invention and comprise a typical batch system with the exception that the substrate is added in increments as the fermentation progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch fermentations are common and well known in the art and examples may be found in Thomas D. Brock in Biotechnology: A Textbook of Industrial Microbiology, Second Edition (1989) Sinauer Associates, Inc., Sunderland, Mass. (hereinafter “Brock”), or Deshpande, Mukund V., Appl. Biochem. Biotechnol., 36:227, (1992).

Although it is common to produce fusion peptides in batch mode, it is contemplated that the method would be adaptable to continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

Applicants specifically incorporate the entire contents of all cited references in this disclosure. Further, when an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmol” means micromole(s), “pmol” means picomole(s), “g” means gram(s), “μg” means microgram(s), “mg” means milligram(s), “g” means the gravitation constant, “rpm” means revolutions per minute, “DTT” means dithiothreitol, and “cat#” means catalog number.

### General Methods

Standard recombinant DNA and molecular cloning techniques used in the Examples are well known in the art and are described by Maniatis, (supra); Silhavy et al., (supra); and Ausubel et al., (supra).

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or in Brock (supra). All reagents, restriction enzymes and materials used for the growth and maintenance of bacterial cells were obtained from BD Diagnostic Systems (Sparks, Md.), Invitrogen (Carlsbad, Calif.), Life Technologies (Rockville, Md.), QIAGEN (Valencia, Calif.) or Sigma-Aldrich Chemical Company (St. Louis, Mo.), unless otherwise specified.

### Example 1

### Preparation of Plasmid pLX121 for Evaluating Inclusion Body Tag Performance

A genetic construct was prepared for evaluating the performance of the present inclusion body tags when fused to a soluble peptide of interest. The peptide of interest used in the present examples was prepared from a previously reported peptide-based triblock dispersant (U.S. Ser. No. 10/935,254).

### Cloning of the TBP1 Gene

The TBP1 gene, encoding the TBP1 peptide, was selected for evaluation of the present inclusion body tags. The synthetic TBP1 peptide is peptide-based triblock dispersant comprising a carbon-black binding domain, a hydrophilic peptide linker, and a cellulose binding domain (see. Example 15 of U.S. patent application Ser. No. 10/935,254).

The TBP1 gene (SEQ ID NO: 1) encoding the 68 amino acid peptide TBP101 (SEQ ID NO: 2) was assembled from synthetic oligonucleotides (Sigma-Genosys, Woodlands, Tex.; Table 1).

TABLE 1
Oligonucleotides Used to Prepare the TBP1

| | | SEQ |
|---|---|---|
| Oligonucleotide | | ID |
| Name | Nucleotide Sequence (5′-3′) | NO: |
| TBP1(+)1 | GGATCCATCGAAGGTCGTTTCCACGAA | 3 |
| | AACTGGCCGTCTGGTGGCGGTACCTC | |
| | TACTTCCAAAGCTTCCACCACTACGAC | |
| | TTCTAGCAAAACCACCACTACAT | |
| TBP1(+)2 | CCTCTAAGACTACCACGACTACCTCCAA | 4 |
| | AACCTCTACTACCTCTAGCTCCTCTACG | |
| | GGCGGTGGCACTCACAAGACCTCTACTC | |
| | AGCGTCTGCTGGCTGCATAA | |
| TBP1(−)1 | TTATGCAGCCAGCAGACGCTGAGTAGAG | 5 |
| | GTCTTGTGAGTGCCACCGCCCGTAGAG | |
| | GAGCTAGAGGTAGT | |
| TBP1(−)2 | AGAGGTTTTGGAGGTAGTCGTGGTAGTC | 6 |
| | TTAGAGGATGTAGTGGTGGTTTTGCTAG | |
| | AAGTCGTAGTGGT | |
| TBP1(−)3 | GGAAGCTTTGGAAGTAGAGGTACCGC | 7 |
| | CACCAGACGGCCAGTTTTCGTGGAAAC | |
| | GACCTTCGATGGATCC | |

Each oligonucleotide was phosphorylated with ATP using T4 polynucleotide kinase. The resulting oligonucleotides were mixed, boiled for 5 min, and then cooled to room temperature slowly. Finally, the annealed oligonucleotides were ligated with T4 DNA ligase to give synthetic DNA fragment TBP1, given as SEQ ID NO: 1.

Construction of pINK101 Expression Plasmid:

Lambda phage site-specific recombination was used for preparation and expression of the present fusion proteins (Gateway™ System; Invitrogen, Carlsbad, Calif.). TBP1 was integrated into the Gateway™ system for protein over-expression. In the first step, 2 μL of the TBP1 ligation mixture was used in a 50-μL PCR reaction. Reactions were catalyzed by pfu DNA polymerase (Stratagene, La Jolla, Calif.), following the standard PCR protocol. Primer 5′TBP1 (5′-CACCGGATCCATCGAAGGTCGT-3′; SEQ ID NO: 8) and 3′TBP1 (5′-TCATTATGCAGCCAGCAGCGC-3′; SEQ ID NO: 9) were used for amplification of the TBP1 fragment. Due to the design of these primers, an additional sequence of CACC and another stop codon TGA were added to the 5′ and 3′ ends of the amplified fragments.

The amplified TBP1 was directly cloned into pENTR™/D-TOPO® vector (SEQ ID NO: 10) using Invitrogen's pENTR™ directional TOPO® cloning kit (Invitrogen; Catalog K2400-20), resulting in the Gateway™ entry plasmid pENTR-TBP1. This entry plasmid was propagated in One Shot® TOP10 E. coli cells (Invitrogen). The accuracy of the PCR amplification and cloning procedures were confirmed by DNA sequencing analysis. The entry plasmid was mixed with pDEST17 (Invitrogen, SEQ ID NO: 11). LR recombination reactions were catalyzed by LR Clonase™ (Invitrogen). The destination plasmid, pINK101 was constructed and propagated in the DH5α E. coli strain. The accuracy of the recombination reaction was determined by DNA sequencing. All reagents for LR recombination reactions (i.e., lambda phage site-specific recombination) were provided in Invitrogen's E. coli expression system with the Gateway™ Technology kit. The site-specific recombination process followed the manufacturer's instructions (Invitrogen).

The resulting plasmid, named pINK101, contains the coding regions for recombinant protein 6H-TBP1, named INK101 (SEQ ID NOs 12 and 13), which is an 11.6 kDa protein. The protein sequence includes a 6×His tag and a 24 amino acid linker that includes Factor Xa protease recognition site before the sequence of the TBP101 peptide.

The amino acid coding region for the 6×His tag and the following linker comprising the Factor Xa protease recognition site were excised from pINK101 by digestion with the NdeI and BamHI restriction enzymes.

The TBP1 gene (SEQ ID NO: 1) encodes a polypeptide (SEQ ID NO: 2) having a ST linker flanked by Gly-Gly-Gly amino acids. The system was made more modular by further mutagenesis to change the upstream amino acid sequence from Gly-Gly-Gly to Ala-Gly-Gly (codon GGT changed to GCC) and the downstream Gly-Gly-Gly to Gly-Gly-Ala (codon GGT GGC changed to GGC GCC). These changes provided a NgoMI restriction site and a KasI restriction site flanking the ST linker, thus facilitating replacement of any element in TBP1.

Further modifications were made to TBP101 including the addition of an acid cleavable site to facilitate the removal of any tag sequence encoded by the region between the NdeI and BamHI sites of the expression plasmid. The resulting plasmid was called pLX121 (also referred to as “pINK101DP”; SEQ ID NO: 14). These modifications changed the amino acids E-G to D-P (acid cleavable aspartic acid—proline linkage) using the Stratagene QuikChange® II Site-Directed Mutagenesis Kit Cat# 200523 (La Jolla, Calif.) as per the manufacturer's protocol using the primers INK101+ (5′-CCCCTTCACCGGATCCATCGATCCACGTTTCCACGAAAACTGGCC-3′; SEQ ID 15) and INK101− (5′-GGCCAGTTTTCGTGGAAACGTGGATCGATGGATCCGGTGAAGGGG-3′; SEQ ID NO 16). The sequences were confirmed by DNA sequence analysis. The coding region and the corresponding amino acid sequence of the modified protein, INK101DP, is provided as SEQ ID NOs 17 and 18, respectively. INK101DP (also referred to herein as “TBP101 DP”) was used to evaluate the present inclusion body tags.

INK101DP Peptide

(SEQ ID NO: 18)

MSYYHHHHHHLESTSLYKKAGSAAAPFTGSIDPRFHENWPSAGGTSTS
KASSSKTTTTSSKTTTTTSKTSTTSSSSTGGATHKTSTQRLLAA

The aspartic acid—proline acid cleavable linker is bolded. The DP linker moiety replaced the EG moiety found in the unmodified TBP101 peptide (SEQ ID NO: 2). The modified TBP101 peptide (i.e., peptide of interest) is underlined.

### Example 2

### Generation of Zein-Based Inclusion Body Tag Library

Several series of inclusion body tag libraries were generated from the Zea mays zein storage protein (GenBank® Accession No. AAP32017; SEQ ID NO: 20 encoded by the coding sequence as represented by SEQ ID NO:19). Three series of putative inclusion body tags (typically 15 amino acids in length) were prepared from 15 amino acid segments of the zein protein. Library series #1 (IBTs 65-79) was prepared from creating a set of 15 amino acid long peptides spanning the entire length of the zein protein starting with amino acid residue position 1 of SEQ ID NO: 20 (i.e. IBT-65=amino acid residues 1-15 of SEQ ID NO: 20, IBT-66=amino acid residues 16-30 of SEQ ID NO: 2,). Library series #2 (IBTs 80-121) was prepared in a similar fashion, except that the first member of the library series started with amino acid residue position 6 of SEQ ID NO: 20. Library series #3 (IBTs 122-135) was also prepared in a similar fashion starting at amino acid position 11 of SEQ ID NO: 20. In this way, an overlapping library 15 amino acid long peptides were prepared that spanned the entire length of zein protein (Table 2).

Based on the expression ranking data (i.e. the ability of the inclusion body tag to induce insoluble fusion protein when fused to a normally soluble peptide of interest), several addition inclusion body tags (IBTs 158-159) of varying in length were prepared from regions of the zein protein suitable for use as inclusion body tags (Table 2).

The inclusion body tags were assembled from two complementary synthetic E. coli biased oligonucleotides (Sigma Genosys). Overhangs were included in each oligonucleotide to generate cohesive ends compatible with the restriction sites NdeI and BamHI.

The oligonucleotides (Table 2) were annealed by combining 100 pmol of each oligonucleotide in deionized water into one tube and heated in a water bath set at 99° C. for 10 minutes after which the water bath was turned off. The oligonucleotides were allowed to anneal slowly until the water bath reached room temperature (20-25° C.). The annealed oligonucleotides were diluted in 100 μL water prior to ligation into the test vector. The vector pLX121 (SEQ ID NO: 14) comprises the open reading frame encoding the INK101DP peptide (SEQ ID NO: 18). The vector was digested in Buffer 2 (New England Biolabs, Beverly Mass.) comprising 10 mM Tris-HCl, 10 mM MgCl₂, 50 mM NaCl, 1 mM dithiothreitol (DTT); pH ˜7.9) with the NdeI and BamHI restriction enzymes to release a 90 by fragment corresponding to the original His6 containing inclusion body fusion partner and the linker from the parental pDEST17 plasmid that includes the att site of the Gateway™ Cloning System. The NdeI-BamHI fragments from the digested plasmid were separated by agarose gel electrophoresis and the vector was purified from the gel by using Qiagen QIAquick® Gel Extraction Kit (QIAGEN Valencia, Calif.; cat# 28704).

The diluted and annealed oligonucleotides (approximately 0.2 pmol) were ligated with T4 DNA Ligase (New England Biolabs Beverly, Mass.; catalog #M0202) to NdeI-BamHI digested, gel purified, plasmid pLX121 (approximately 50 ng) at 12° C. for 18 hours. DNA sequence analysis confirmed the expected plasmid sequence.

TABLE 2
Oligonucleotide Sequences Used to Prepare the Various
Zein-Based Inclusion Body Tags (IBTs)

| | | | | Amino Acid |
|---|---|---|---|---|
| | | | | Residue |
| | | | IBT Amino | Positions of the |
| | | | Acid | Zein Protein |
| Inclusion | DNA | Oligonucleotide | Sequence | (SEQ ID NO: |
| Body Tag | strand | (SEQ ID NO.) | (SEQ ID NO.) | 20) |
| IBT-65 | + | 21 | 111 | 1-15 |
| IBT-65 | − | 22 | | |
| IBT-66 | + | 23 | 112 | 16-30 |
| IBT-66 | − | 24 | | |
| IBT-67 | + | 25 | 113 | 31-45 |
| IBT-67 | − | 26 | | |
| IBT-68 | + | 27 | 114 | 46-60 |
| IBT-68 | − | 28 | | |
| IBT-69 | + | 29 | 115 | 61-75 |
| IBT-69 | − | 30 | | |
| IBT-70 | + | 31 | 116 | 76-90 |
| IBT-70 | − | 32 | | |
| IBT-71 | + | 33 | 117 | 91-105 |
| IBT-71 | − | 34 | | |
| IBT-72 | + | 35 | 118 | 106-120 |
| IBT-72 | − | 36 | | |
| IBT-73 | + | 37 | 119 | 121-135 |
| IBT-73 | − | 38 | | |
| IBT-74 | + | 39 | 120 | 136-150 |
| IBT-74 | − | 40 | | |
| IBT-75 | + | 41 | 121 | 151-165 |
| IBT-75 | − | 42 | | |
| IBT-76 | + | 43 | 122 | 166-180 |
| IBT-76 | − | 44 | | |
| IBT-77 | + | 45 | 123 | 181-195 |
| IBT-77 | − | 46 | | |
| IBT-78 | + | 47 | 124 | 196-210 |
| IBT-78 | − | 48 | | |
| IBT-79 | + | 49 | 125 | 211-223 |
| IBT-79 | − | 50 | | |
| IBT-108 | + | 51 | 126 | 6-20 |
| IBT-108 | − | 52 | | |
| IBT-109 | + | 53 | 127 | 21-35 |
| IBT-109 | − | 54 | | |
| IBT-110 | + | 55 | 128 | 36-50 |
| IBT-110 | − | 56 | | |
| IBT-111 | + | 57 | 129 | 51-65 |
| IBT-111 | − | 58 | | |
| IBT-112 | + | 59 | 130 | 66-80 |
| IBT-112 | − | 60 | | |
| IBT-113 | + | 61 | 131 | 81-95 |
| IBT-113 | − | 62 | | |
| IBT-114 | + | 63 | 132 | 96-110 |
| IBT-114 | − | 64 | | |
| IBT-115 | + | 65 | 133 | 111-125 |
| IBT-115 | − | 66 | | |
| IBT-116 | + | 67 | 134 | 126-140 |
| IBT-116 | − | 68 | | |
| IBT-117 | + | 69 | 135 | 141-155 |
| IBT-117 | − | 70 | | |
| IBT-118 | + | 71 | 136 | 156-170 |
| IBT-118 | − | 72 | | |
| IBT-119 | + | 73 | 137 | 171-185 |
| IBT-119 | − | 74 | | |
| IBT-120 | + | 75 | 138 | 186-200 |
| IBT-120 | − | 76 | | |
| IBT-121 | + | 77 | 139 | 201-215 |
| IBT-121 | − | 78 | | |
| IBT-122 | + | 79 | 140 | 11-25 |
| IBT-122 | − | 80 | | |
| IBT-123 | + | 81 | 141 | 26-40 |
| IBT-123 | − | 82 | | |
| IBT-124 | + | 83 | 142 | 41-55 |
| IBT-124 | − | 84 | | |
| IBT-125 | + | 85 | 143 | 56-70 |
| IBT-125 | − | 86 | | |
| IBT-126 | + | 87 | 144 | 71-85 |
| IBT-126 | − | 88 | | |
| IBT-127 | + | 89 | 145 | 86-100 |
| IBT-127 | − | 90 | | |
| IBT-128 | + | 91 | 146 | 101-115 |
| IBT-128 | − | 92 | | |
| IBT-129 | + | 93 | 147 | 116-130 |
| IBT-129 | − | 94 | | |
| IBT-130 | + | 95 | 148 | 131-145 |
| IBT-130 | − | 96 | | |
| IBT-131 | + | 97 | 149 | 146-160 |
| IBT-131 | − | 98 | | |
| IBT-132 | + | 99 | 150 | 161-175 |
| IBT-132 | − | 100 | | |
| IBT-133 | + | 101 | 151 | 176-190 |
| IBT-133 | − | 102 | | |
| IBT-134 | + | 103 | 152 | 191-205 |
| IBT-134 | − | 104 | | |
| IBT-135 | + | 105 | 153 | 206-220 |
| IBT-135 | − | 106 | | |
| IBT-158 | + | 107 | 154 | 86-110 |
| IBT-158 | − | 108 | | |
| IBT-159 | + | 109 | 155 | 91-110 |
| IBT-159 | − | 110 | | |

The resulting expression vectors were individually transformed into the arabinose inducible expression strain E. coli BL21-Al (Invitrogen; cat#C6070-03).

### Transformation and Expression

Each expression vector was individually transferred into BL21-Al chemically competent E. coli cells for expression analysis. To produce the recombinant protein, 3 mL of LB-ampicillin broth (10 g/L bacto-tryptone, 5 g/L bacto-yeast extract, 10 g/L NaCl, 100 mg/L ampicillin; pH 7.0) was inoculated with one colony of the transformed bacteria and the culture was shaken at 37° C. until the OD₆₀₀ reached 0.6. Expression was induced by adding 0.03 mL of 20% L-arabinose (final concentration 0.2%, Sigma-Aldrich, St. Louis, Mo.) to the culture and shaking was continued for another 3 hours. For whole cell analysis, 0.1 OD₆₀₀ mL of cells were collected, pelleted, and 0.06 mL SDS PAGE sample buffer (1×LDS Sample Buffer (Invitrogen cat# NP0007), 6 M urea, 100 mM DTT) was added directly to the whole cells. The samples were heated at 99° C. for 10 minutes to solubilize the proteins. The solubilized proteins were then loaded onto 4-12% gradient MES NuPAGE® gels (NuPAGE® gels cat #NP0322, MES Buffer cat#NP0002; Invitrogen) and visualized with a Coomassie® G-250 stain (SimplyBlue™ SafeStain; Invitrogen; cat#LC6060).

### Example 3

Verification of Zein-Based Peptide Tags for Inclusion Body Formation

To verify that the fusion partner drove expression into insoluble inclusion bodies, it was necessary to lyse the collected cells (0.1 OD₆₀₀ mL of cells) and fractionate the insoluble from the soluble fraction by centrifugation. Cells were lysed using CelLytic™ Express (Sigma, St. Louis, Mo. cat#C-1990) according to the manufacturer's instructions. Cells that do not produce inclusion bodies undergo complete lysis and yielded a clear solution. Cells expressing inclusion bodies appeared turbid even after complete lysis.

The method used to rank all inclusion body tags was a subjective visual inspection of SimplyBlue™ SafeStain stained PAGE gels. The scoring system was 0, 1, 2 or 3. If no band is detected then a zero score is given. A score of three is given to very heavily stained wide expressed bands. Bands that are weak are scored a one and moderate bands are scored a two. Any score above zero indicated the presence of inclusion bodies (Table 4).

Soluble and insoluble fractions were separated by centrifugation and analyzed by polyacrylamide gel electrophoresis and visualized with SimplyBlue™ SafeStain. Analysis of the cell protein by polyacrylamide gel electrophoresis was used to detect the production of the fusion protein in the whole cell and insoluble fractions but not the soluble cell fraction. Several fusion proteins comprising a 15 amino acid long inclusion body tag derived from amino acid residues 76-175 of SEQ ID NO: 20 were found to be insoluble. This result suggested that it was possible to have very small fusion partners (at least 15 amino acids in length) to facilitate production of peptides in inclusion bodies (Table 3)

TABLE 3
Zein-Based Inclusion Body Tag Expression
Ranking

| | Zein-based Inclusion | |
|---|---|---|
| | Body Tag Amino | |
| IBT | Acid Sequence | Expression |
| Designation | (SEQ ID NO:) | Ranking |
| IBT 65 | MRVLLVALALLALAA | 0 |
| | (SEQ ID NO: 111) | |
| IBT 66 | SATSTHTSGGCGCQP | 0 |
| | (SEQ ID NO: 112) | |
| IBT 67 | PPPVHLPPPVHLPPP | 0 |
| | (SEQ ID NO: 113) | |
| IBT 68 | VHLPPPVHLPPPVHL | 0 |
| | (SEQ ID NO: 114) | |
| IBT 69 | PPPVHLPPPVHVPPP | 0 |
| | (SEQ ID NO: 115) | |
| IBT 70 | VHLPPPPCHYPTQ | 2 |
| | (SEQ ID NO: 116) | |
| IBT 71 | RPQPHPQPHPCPCQQ | 3 |
| | (SEQ ID NO: 117) | |
| IBT 72 | PHPSPCQLQGTCGVG | 0 |
| | (SEQ ID NO: 118) | |
| IBT 73 | STPILGQCVEFLRHQ | 2 |
| | (SEQ ID NO: 119) | |
| IBT 74 | CSPTATPYCSPQCQS | 0 |
| | (SEQ ID NO: 120) | |
| IBT 75 | LRQQCCQQLRQVEPQ | 1 |
| | (SEQ ID NO: 121) | |
| IBT 76 | HRYQAIFGLVLQSIL | 0 |
| | (SEQ ID NO: 122) | |
| IBT 77 | QQQPQSGQVAGLLAA | 0 |
| | (SEQ ID NO: 123) | |
| IBT 78 | QIAQQLTAMCGLQQP | 0 |
| | (SEQ ID NO: 124) | |
| IBT 79 | TPCPYAAAGGVPH | 1 |
| | (SEQ ID NO: 125) | |
| IBT 108 | VALALLALAASATST | 0 |
| | (SEQ ID NO: 126) | |
| IBT 109 | HTSGGCGCQPPPPVH | 0 |
| | (SEQ ID NO: 127) | |
| IBT 110 | LPPPVHLPPPVHLPP | 0 |
| | (SEQ ID NO: 128) | |
| IBT 111 | PVHLPPPVHLPPPVH | 0 |
| | (SEQ ID NO: 129) | |
| IBT 112 | LPPPVHVPPPVHLPP | 0 |
| | (SEQ ID NO: 130) | |
| IBT 113 | PPCHYPTQPPRPQPH | 3 |
| | (SEQ ID NO: 131) | |
| IBT 114 | PQPHPCPCQQPHPSP | 2 |
| | (SEQ ID NO: 132) | |
| IBT 115 | CQLQGTCGVGSTPIL | 1 |
| | (SEQ ID NO: 133) | |
| IBT 116 | GQCVEFLRHQCSPTA | 0 |
| | (SEQ ID NO: 134) | |
| IBT 117 | TPYCSPQCQSLRQQC | 1 |
| | (SEQ ID NO: 135) | |
| IBT 118 | CQQLRQVEPQHRYQA | 0 |
| | (SEQ ID NO: 136) | |
| IBT 119 | IFGLVLQSILQQQPQ | 0 |
| | (SEQ ID NO: 137) | |
| IBT 120 | SGQVAGLLAAQIAQQ | 0 |
| | (SEQ ID NO: 138) | |
| IBT 121 | LTAMCGLQQPTPCPY | 0 |
| | (SEQ ID NO: 139) | |
| IBT 122 | LALAASATSTHTSGG | 0 |
| | (SEQ ID NO: 140) | |
| IBT 123 | CGCQPPPPVHLPPPV | 0 |
| | (SEQ ID NO: 141) | |
| IBT 124 | HLPPPVHLPPPVHLP | 0 |
| | (SEQ ID NO: 142) | |
| IBT 125 | PPVHLPPPVHLPPPV | 0 |
| | (SEQ ID NO: 143) | |
| IBT 126 | HVPPPVHLPPPPCHY | 0 |
| | (SEQ ID NO: 144) | |
| IBT 127 | PTQPPRPQPHPQPHP | 3 |
| | (SEQ ID NO: 145) | |
| IBT 128 | CPCQQPHPSPCQLQG | 0 |
| | (SEQ ID NO: 146) | |
| IBT 129 | TCGVGSTPILGQCVE | 1 |
| | (SEQ ID NO: 147) | |
| IBT 130 | FLRHQCSPTATPYCS | 3 |
| | (SEQ ID NO: 148) | |
| IBT 131 | PQCQSLRQQCCQQLR | 2 |
| | (SEQ ID NO: 149) | |
| IBT 132 | QVEPQHRYQAIFGLV | 1 |
| | (SEQ ID NO: 150) | |
| IBT 133 | LQSILQQQPQSGQVA | 0 |
| | (SEQ ID NO: 151) | |
| IBT 134 | GLLAAQIAQQLTAMC | 0 |
| | (SEQ ID NO: 152) | |
| IBT 135 | GLQQPTPCPYAAAGG | 0 |
| | (SEQ ID NO: 153) | |
| IBT 158 | PTQPPRPQPHPQPHPCPCQQPHPSP | 2 |
| | (SEQ ID NO: 154) | |
| IBT 159 | RPQPHPQPHPCPCQQPHPSP | 2 |
| | (SEQ ID NO: 155) | |

### Example 4

### Synthesis, Cloning, and Evaluation of Fusion Peptides Comprising

Inclusion Body Tags IBT-180 and IBT-181 The expression ranking data from the various zein-based inclusion body tags was evaluated and used to design two additional inclusion body tags (IBT-180 and IBT-181) comprising a T7 translational enhancer (MASMTGGQQMG; SEQ ID NO: 156) linked to the N-terminal portion of an inclusion body forming region of the zein protein. As used herein, “T7 translational enhancer element” means the N-terminal coding sequence of bacteriophage T7 gene 10 (Rosenberg, A H et al., Gene 56:125-135 (1987)), which provides a standardized sequence at the critical translation initiation site in the genes encoding the inclusion body tags.

### Design of Inclusion Body Tags IBT-180 and IBT-181

An alignment of the inclusion body tags exhibiting inclusion body forming ability was performed against the zein protein. The initial library of overlapping inclusion body tags was designed span the entire length of the zein protein. Based on the overlapping nature of the inclusion body tag library, every amino acid had up to three opportunities to be in a tag. Relative scores were assigned to each amino acid within the zein protein based on the frequency of occurrence within a peptide tag capable of inducing inclusion body formation. The relative scores were used to assign a final activity score for each amino acid. When activity score for each amino acid was plotted over the length of the scanned protein, a topographical-like map was generated depicting the ability of certain domains on the scanned protein to induce inclusion body formation. From this assessment, it was determined that inclusion body tags prepared from the region of the zein protein encompassed by amino acid residues 76-175 of SEQ ID NO: 20 was particularly effective in inducing inclusion body formation.

A 100 amino acid long functional inclusion body tag, IBT-181 (SEQ ID NO: 158), comprising amino acid residues 76 to 175 of SEQ ID NO: 20 and a shorter 30 amino acid inclusion body tag, IBT-180 (SEQ ID NO: 157), comprising a subset of this region (amino acid residues 76 to 105 of SEQ ID NO: 20) were prepared. Both tags also included a short 11 amino acid T7 tag (a translational enhancer) (MASMTGGQQMG; SEQ ID NO: 156) added to the N-terminus of each tag.

### Synthesis and Cloning Procedure of IBT-180 and IBT-181

The nucleic acid molecules encoding the inclusion body tags IBT-180 (SEQ ID NO: 157) and IBT-181 (SEQ ID NO: 158) were synthesized and delivered as plasmids harboring kanamycin resistance by DNA 2.0 Inc. (Menlo Park, Calif.). The nucleotide sequence encoding each inclusion body tag was flanked by NdeI and BamHI restriction sites.

The vector comprising the nucleic acid molecule encoding the IBT-180 tag was digested in Buffer 2 (New England Biolabs 10 mM Tris-HCl, 10 mM MgCl₂, 50 mM NaCl, 1 mM dithiothreitol; pH7.9) with the NdeI and BamHI restriction enzymes (New England Biolabs Beverly, Mass.). Likewise, the test system expression vector pLX121 (SEQ ID NO: 14) was digested with NdeI and BamHI as described in the previous examples. The IBT-180 inclusion body tag restriction digest was directly ligated to the NdeI/BamHI digested test expression vector pLX121 with T4 DNA Ligase (New England Biolabs Beverly, Mass. cat#M0202) at 12° C. for 18 hours. Ampicillin resistant colonies were sequenced. The sequence of the plasmid (pLX363) was confirmed. Expression plasmid pLX363 comprises the chimeric gene encoding the IBT 180 tagged fusion protein, operably linked to an arabinose inducible promoter.

Inclusion body tag IBT-181 (SEQ ID BO: 158) was cloned using the same procedure as described for IBT-180, resulting in the expression plasmid pLX364. Expression plasmid pLX364 comprises the chimeric gene encoding the IBT 181 tagged fusion protein operably linked to an arabinose inducible promoter.

### Transformation and Expression of IBT-180 and IBT-181

Expression plasmids pLX363 and pLX364 were transformed, expressed, and evaluated using the procedures described in Examples 2 and 3. The expression ranking results are provided in Table 4.

TABLE 4
Inclusion Body Tag Expression Ranking for
IBT-180 and IBT-181

| | Zein-based Inclusion | |
|---|---|---|
| | Body Tag Amino | |
| IBT | Acid Sequence | Expression |
| Designation | (SEQ ID NO:) | Ranking |
| IBT 180 | MASMTGGQQMGVHLPPPPCHY | 2 |
| | PTQPPRPQPHPQPHPCPCQQ | |
| | (SEQ ID NO: 157) | |
| IBT 181 | MASMTGGQQMGVHLPPPPCHY | 2 |
| | PTQPPRPQPHPQPHPCPCQQPH | |
| | PSPCQLQGTCGVGSTPILGQCVE | |
| | FLRHQCSPTATPYCSPQCQSLR | |
| | QQCCQQLRQVEPQHRYQAIFGL | |
| | V | |
| | (SEQ ID NO: 158) | |

### Example 5

### Generation of Cystatin-Based Inclusion Body Tag Library

Several series of inclusion body tag libraries were generated from the 133 amino acid Daucus carota cystatin protein (GenBank® Accession No. BAA20464; SEQ ID NO: 160 encoded by the coding sequence as represented by SEQ ID NO: 159). Three series of putative inclusion body tags (typically 12 or 13 amino acids in length) were prepared from various portions of the cystatin protein. Library series #1 (IBTs 141-151) was prepared from creating a set of 12 or 13 amino acid long peptides spanning the entire length of the cystatin protein starting with amino acid residue position 1 of SEQ ID NO: 160 (i.e. IBT-141=amino acid residues 1-12 of SEQ ID NO: 160, IBT-142=amino acid residues 13-24 of SEQ ID NO: 160, etc.). Library series #2 (IBTs 160-169) was prepared in a similar fashion, except that the first member of the library series started with amino acid residue position 5 of SEQ ID NO: 160. Library series #3 (IBTs 170-179) was also prepared in a similar fashion starting at amino acid position 9 of SEQ ID NO: 160. In this way, an overlapping library 12 or 13 amino acid long peptides were prepared that spanned the entire length of the cystatin protein (Table 5).

The inclusion body tags were assembled from two complementary synthetic E. coli biased oligonucleotides (Sigma Genosys). Overhangs were included in each oligonucleotide to generate cohesive ends compatible with the restriction sites NdeI and BamHI.

The oligonucleotides (Table 5) were annealed by combining 100 pmol of each oligonucleotide in deionized water into one tube and heated in a water bath set at 99° C. for 10 minutes after which the water bath was turned off. The oligonucleotides were allowed to anneal slowly until the water bath reached room temperature (20-25° C.). The annealed oligonucleotides were diluted in 100 μl water prior to ligation into the test vector. The vector pLX121 (SEQ ID NO: 14) comprises the open reading frame encoding the INK101DP peptide (SEQ ID NO: 18). The vector was digested in Buffer 2 (New England Biolabs, Beverly Mass.) comprising 10 mM Tris-HCl, 10 mM MgCl₂, 50 mM NaCl, 1 mM dithiothreitol (DTT); pH ˜7.9) with the NdeI and BamHI restriction enzymes to release a 90 by fragment corresponding to the original His6 containing inclusion body fusion partner and the linker from the parental pDEST17 plasmid that includes the att site of the Gateway™ Cloning System. The NdeI-BamHI fragments from the digested plasmid were separated by agarose gel electrophoresis and the vector was purified from the gel by using Qiagen QIAquick® Gel Extraction Kit (QIAGEN Valencia, Calif.; cat# 28704).

The diluted and annealed oligonucleotides (approximately 0.2 μmol) were ligated with T4 DNA Ligase (New England Biolabs Beverly, Mass.; catalog #M0202) to NdeI-BamHI digested, gel purified, plasmid pLX121 (approximately 50 ng) at 12° C. for 18 hours. DNA sequence analysis confirmed the expected plasmid sequence.

TABLE 5
Oligonucleotide Sequences Used to Prepare the Various
Cystatin-Based Inclusion Body Tags (IBTs)

| | | | | Amino Acid |
|---|---|---|---|---|
| | | | | Residue |
| | | | | Positions |
| | | | IBT Amino | of the Cystatin |
| | | | Acid | protein |
| Inclusion | DNA | Oligonucleotide | Sequence | (SEQ ID NO: |
| Body Tag | strand | (SEQ ID NO.) | (SEQ ID NO.) | 160) |
| IBT-141 | + | 161 | 223 | 1-12 |
| IBT-141 | − | 162 | | |
| IBT-142 | + | 163 | 224 | 13-24 |
| IBT-142 | − | 164 | | |
| IBT-143 | + | 165 | 225 | 25-36 |
| IBT-143 | − | 166 | | |
| IBT-144 | + | 167 | 226 | 37-48 |
| IBT-144 | − | 168 | | |
| IBT-145 | + | 169 | 227 | 49-60 |
| IBT-145 | − | 170 | | |
| IBT-146 | + | 171 | 228 | 61-72 |
| IBT-146 | − | 172 | | |
| IBT-147 | + | 173 | 229 | 73-84 |
| IBT-147 | − | 174 | | |
| IBT-148 | + | 175 | 230 | 85-96 |
| IBT-148 | − | 176 | | |
| IBT-149 | + | 177 | 231 | 97-108 |
| IBT-149 | − | 178 | | |
| IBT-150 | + | 179 | 232 | 109-120 |
| IBT-150 | − | 180 | | |
| IBT-151 | + | 181 | 233 | 121-133 |
| IBT-151 | − | 182 | | |
| IBT-160 | + | 183 | 234 | 5-16 |
| IBT-160 | − | 184 | | |
| IBT-161 | + | 185 | 235 | 17-28 |
| IBT-161 | − | 186 | | |
| IBT-162 | + | 187 | 236 | 29-40 |
| IBT-162 | − | 188 | | |
| IBT-163 | + | 189 | 237 | 41-52 |
| IBT-163 | − | 190 | | |
| IBT-164 | + | 191 | 238 | 53-64 |
| IBT-164 | − | 192 | | |
| IBT-165 | + | 193 | 239 | 65-76 |
| IBT-165 | − | 194 | | |
| IBT-166 | + | 195 | 240 | 77-88 |
| IBT-166 | − | 196 | | |
| IBT-167 | + | 197 | 241 | 89-100 |
| IBT-167 | − | 198 | | |
| IBT-168 | + | 199 | 242 | 101-112 |
| IBT-168 | − | 200 | | |
| IBT-169 | + | 201 | 243 | 113-124 |
| IBT-169 | − | 202 | | |
| IBT-170 | + | 203 | 244 | 9-20 |
| IBT-170 | − | 204 | | |
| IBT-171 | + | 205 | 245 | 21-32 |
| IBT-171 | − | 206 | | |
| IBT-172 | + | 207 | 246 | 33-44 |
| IBT-172 | − | 208 | | |
| IBT-173 | + | 209 | 247 | 45-56 |
| IBT-173 | − | 210 | | |
| IBT-174 | + | 211 | 248 | 57-68 |
| IBT-174 | − | 212 | | |
| IBT-175 | + | 213 | 249 | 69-80 |
| IBT-175 | − | 214 | | |
| IBT-176 | + | 215 | 250 | 81-92 |
| IBT-176 | − | 216 | | |
| IBT-177 | + | 217 | 251 | 93-104 |
| IBT-177 | − | 218 | | |
| IBT-178 | + | 219 | 252 | 105-116 |
| IBT-178 | − | 220 | | |
| IBT-179 | + | 221 | 253 | 117-128 |
| IBT-179 | − | 222 | | |

The resulting expression vectors were individually transformed into the arabinose inducible expression strain E. coli BL21-Al (Invitrogen; cat#C6070-03).

### Transformation and Expression

Each expression vector was individually transferred into BL21-Al chemically competent E. coli cells for expression analysis. To produce the recombinant protein, 3 mL of LB-ampicillin broth (10 g/L bacto-tryptone, 5 g/L bacto-yeast extract, 10 g/L NaCl, 100 mg/L ampicillin; pH 7.0) was inoculated with one colony of the transformed bacteria and the culture was shaken at 37° C. until the OD₆₀₀ reached 0.6. Expression was induced by adding 0.03 mL of 20% L-arabinose (final concentration 0.2%, Sigma-Aldrich, St. Louis, Mo.) to the culture and shaking was continued for another 3 hours. For whole cell analysis, 0.1 OD₆₀₀ mL of cells were collected, pelleted, and 0.06 mL SDS PAGE sample buffer (1×LDS Sample Buffer (Invitrogen cat# NP0007), 6 M urea, 100 mM DTT) was added directly to the whole cells. The samples were heated at 99° C. for 10 minutes to solubilize the proteins. The solubilized proteins were then loaded onto 4-12% gradient MES NuPAGE® gels (NuPAGE® gels cat #NP0322, MES Buffer cat# NP0002; Invitrogen) and visualized with a Coomassie® G-250 stain (SimplyBlue™ SafeStain; Invitrogen; cat# LC6060).

### Example 6

### Verification of Inclusion Body Formation by Cystatin-Based Inclusion Body Tags

To verify that the fusion partner drove expression into insoluble inclusion bodies, it was necessary to lyse the collected cells (0.1 OD₆₀₀ mL of cells) and fractionate the insoluble from the soluble fraction by centrifugation. Cells were lysed using CelLytic™ Express (Sigma, St. Louis, Mo. cat#C-1990) according to the manufacturer's instructions. Cells that do not produce inclusion bodies undergo complete lysis and yielded a clear solution. Cells expressing inclusion bodies appeared turbid even after complete lysis.

The method used to rank all inclusion body tags was a subjective visual inspection of SimplyBlue™ SafeStain stained PAGE gels. The scoring system was 0, 1, 2 or 3. If no band is detected then a zero score is given. A score of three is given to very heavily stained wide expressed bands. Bands that are weak are scored a one and moderate bands are scored a two. Any score above zero indicated the presence of inclusion bodies (Table 6).

Soluble and insoluble fractions were separated by centrifugation and analyzed by polyacrylamide gel electrophoresis and visualized with SimplyBlue™ SafeStain. Analysis of the cell protein by polyacrylamide gel electrophoresis was used to detect the production of the fusion protein in the whole cell and insoluble fractions, but not in the soluble cell fraction. Several fusion proteins comprising a 12 to 13 contiguous amino acid long inclusion body tag derived from SEQ ID NO: 164 were found to be insoluble. This result suggested that it was possible to have very small fusion partners (12-13 amino acids in length) to facilitate production of peptides in inclusion bodies (Table 6)

TABLE 6
Cystatin-based Inclusion Body Tag Expression
Ranking

| | Cystatin-based Inclusion | |
|---|---|---|
| | Body Tag Amino | |
| IBT | Acid Sequence | Expression |
| Designation | (SEQ ID NO:) | Ranking |
| IBT 141 | MAAKTQAILILL | 3 |
| | (SEQ ID NO: 223) | |
| IBT 142 | LISAVLIASPAA | 2 |
| | (SEQ ID NO: 224) | |
| IBT 143 | GLGGSGAVGGRT | 0 |
| | (SEQ ID NO: 225) | |
| IBT 144 | EIPDVESNEEIQ | 0 |
| | (SEQ ID NO: 226) | |
| IBT 145 | QLGEYSVEQYNQ | 1 |
| | (SEQ ID NO: 227) | |
| IBT 146 | QHHNGDGGDSTD | 1 |
| | (SEQ ID NO: 228) | |
| IBT 147 | SAGDLKFVKVVA | 3 |
| | (SEQ ID NO: 229) | |
| IBT 148 | AEKQVVAGIKYY | 3 |
| | (SEQ ID NO: 230) | |
| IBT 149 | LKIVAAKGGHKK | 1 |
| | (SEQ ID NO: 231) | |
| IBT 150 | KFDAEIVVQAWK | 3 |
| | (SEQ ID NO: 232) | |
| IBT 151 | KTKQLMSFAPSHN | 3 |
| | (SEQ ID NO: 233) | |
| IBT 160 | TQAILILLLISA | 0 |
| | (SEQ ID NO: 234) | |
| IBT 161 | VLIASPAAGLGG | 2 |
| | (SEQ ID NO: 235) | |
| IBT 162 | SGAVGGRTEIPD | 0 |
| | (SEQ ID NO: 236) | |
| IBT 163 | VESNEEIQQLGE | 0 |
| | (SEQ ID NO: 237) | |
| IBT 164 | YSVEQYNQQHHN | 1 |
| | (SEQ ID NO: 238) | |
| IBT 165 | GDGGDSTDSAGD | 0 |
| | (SEQ ID NO: 239) | |
| IBT 166 | LKFVKVVAAEKQ | 3 |
| | (SEQ ID NO: 240) | |
| IBT 167 | VVAGIKYYLKIV | 0 |
| | (SEQ ID NO: 241) | |
| IBT 168 | AAKGGHKKKFDA | 2 |
| | (SEQ ID NO: 242) | |
| IBT 169 | EIVVQAWKKTKQ | 0 |
| | (SEQ ID NO: 243) | |
| IBT 170 | LILLLISAVLIA | 0 |
| | (SEQ ID NO: 244) | |
| IBT 171 | SPAAGLGGSGAV | 0 |
| | (SEQ ID NO: 245) | |
| IBT 172 | GGRTEIPDVESN | 0 |
| | (SEQ ID NO: 246) | |
| IBT 173 | EEIQQLGEYSVE | 2 |
| | (SEQ ID NO: 247) | |
| IBT 174 | QYNQQHHNGDGG | 2 |
| | (SEQ ID NO: 248) | |
| IBT 175 | DSTDSAGDLKFV | 2 |
| | (SEQ ID NO: 249) | |
| IBT 176 | KVVAAEKQVVAG | 0 |
| | (SEQ ID NO: 250) | |
| IBT 177 | IKYYLKIVAAKG | 0 |
| | (SEQ ID NO: 251) | |
| IBT 178 | GHKKKFDAEIVV | 3 |
| | (SEQ ID NO: 252) | |
| IBT 179 | QAWKKTKQLMSF | 3 |
| | (SEQ ID NO: 253) | |

What is claimed is:

1. A method for identifying an inclusion body tag from a large insoluble protein comprising:
a) providing a first genetic construct encoding an insoluble full-length protein;
b) constructing a first library of nucleic acid fragments from the first genetic construct of (a), each fragment encoding an inclusion body peptide tag of about 10-50 amino acids such that the peptide tags are generated beginning at the N-terminal region of the peptide and extending to the C-terminal end of the peptide, each peptide tag overlapping with the next peptide tag by about 3 to about 10 amino acids;
c) providing a second genetic construct encoding a target peptide to be expressed in insoluble form;
d) constructing a second library by combining, in combinatorial fashion, the nucleic acid fragments of the first library and the second genetic construct encoding the target peptide to create a library of expressible chimeric constructs; wherein each expressible chimeric construct within the library of expressible chimeric constructs encodes a fusion peptide;
e) transforming host cells with the library of expressible chimeric constructs of (d);
f) growing the transformed host cells of (e) under conditions wherein each expressible chimeric construct is expressed as said fusion peptide
g) selecting the transformed host cells comprising said fusion peptide expressed in insoluble form;
j) identifying the inclusion body tag from the insoluble fusion peptide of (g); and
k) optionally isolating the identified inclusion body tag.

2. The method of claim 1 wherein the insoluble full-length protein is at least 100 amino acids in length and is selected from the group consisting of:
a) a naturally occurring insoluble peptide; and
b) a non-naturally occurring insoluble peptide having at least 70% amino acid identity to the naturally occurring insoluble peptide of (a).

3. The method of claim 1 wherein the inclusion body peptide tag is about 10 to about 35 amino acids in length.

4. The method of claim 3 wherein the inclusion body peptide tag is about 12 to about 15 amino acids in length.

5. The method of claim 1 wherein the overlap between the peptide tags in said first library is about 3 to about 6 amino acids.

6. The method of claim 1 wherein the target peptide to be expressed is selected from the group consisting of a polymer binding peptide, a pigment binding peptide, a hair binding peptide, a nail binding peptide, a skin binding peptide, and an antimicrobial peptide.

7. The method of claim 6 wherein the hair binding peptide is selected from the group consisting of SEQ ID NOs: 262 to 354.

8. The method of claim 6 wherein the skin binding peptide is selected from the group consisting of SEQ ID NOs: 254 to 261.

9. The method of claim 6 wherein the nail binding peptide is selected from the group consisting of SEQ ID NOs: 355 to 356.

10. The method of claim 6 wherein the polymer binding peptide is selected from the group consisting of SEQ ID NOs: 412 to 445.

11. The method of claim 6 wherein the pigment binding peptide is selected from the group consisting of SEQ ID NOs: 386 to 411.

12. The method of claim 6 wherein the antimicrobial peptide is selected from the group consisting of SEQ ID NOs: 357 to 385.

13. The method of claim 1 wherein the host cell is selected from the group consisting of bacteria, yeast and filamentous fungi.

14. The method of claim 13, wherein the host cell is selected from the group consisting of Aspergillus, Trichoderma, Saccharomyces, Pichia, Candida, Hansenula, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus.

15. An inclusion body tag identified by the process of claim 1.
